SEQUENCE LISTING (10 sequences)

SEQ ID NO: 1 - protein, 108 aa, Artificial Sequence
  Synthetic peptide
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 2 - protein, 121 aa, Artificial Sequence
  Synthetic peptide
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 3 - protein, 10 aa, Artificial Sequence
  Synthetic peptide
GFTFSRYWMS

SEQ ID NO: 4 - protein, 17 aa, Artificial Sequence
  Synthetic peptide
NIKQDGSEKYYVDSVKG

SEQ ID NO: 5 - protein, 12 aa, Artificial Sequence
  Synthetic peptide
EGGWFGELAFDY

SEQ ID NO: 6 - protein, 12 aa, Artificial Sequence
  Synthetic peptide
RASQRVSSSYLA

SEQ ID NO: 7 - protein, 7 aa, Artificial Sequence
  Synthetic peptide
DASSRAT

SEQ ID NO: 8 - protein, 9 aa, Artificial Sequence
  Synthetic peptide
QQYGSLPWT

SEQ ID NO: 9 - protein, 176 aa, Homo sapiens
MRIFAVFIFMTYWHLLNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 10 - DNA, 3349 bp, Homo sapiens
ggcgcaacgctgagcagctggcgcgtcccgcgcggccccagttctgcgcagcttcccgaggctccgcaccagccgcgcttctgtccgcctgcagggcattccagaaagatgaggatatttgctgtctttatattcatgacctactggcatttgctgaacgccccatacaacaaaatcaaccaaagaattttggttgtggatccagtcacctctgaacatgaactgacatgtcaggctgagggctaccccaaggccgaagtcatctggacaagcagtgaccatcaagtcctgagtggtaagaccaccaccaccaattccaagagagaggagaagcttttcaatgtgaccagcacactgagaatcaacacaacaactaatgagattttctactgcacttttaggagattagatcctgaggaaaaccatacagctgaattggtcatcccagaactacctctggcacatcctccaaatgaaaggactcacttggtaattctgggagccatcttattatgccttggtgtagcactgacattcatcttccgtttaagaaaagggagaatgatggatgtgaaaaaatgtggcatccaagatacaaactcaaagaagcaaagtgatacacatttggaggagacgtaatccagcattggaacttctgatcttcaagcagggattctcaacctgtggtttaggggttcatcggggctgagcgtgacaagaggaaggaatgggcccgtgggatgcaggcaatgtgggacttaaaaggcccaagcactgaaaatggaacctggcgaaagcagaggaggagaatgaagaaagatggagtcaaacagggagcctggagggagaccttgatactttcaaatgcctgaggggctcatcgacgcctgtgacagggagaaaggatacttctgaacaaggagcctccaagcaaatcatccattgctcatcctaggaagacgggttgagaatccctaatttgagggtcagttcctgcagaagtgccctttgcctccactcaatgcctcaatttgttttctgcatgactgagagtctcagtgttggaacgggacagtatttatgtatgagtttttcctatttattttgagtctgtgaggtcttcttgtcatgtgagtgtggttgtgaatgatttcttttgaagatatattgtagtagatgttacaattttgtcgccaaactaaacttgctgcttaatgatttgctcacatctagtaaaacatggagtatttgtaaggtgcttggtctcctctataactacaagtatacattggaagcataaagatcaaaccgttggttgcataggatgtcacctttatttaacccattaatactctggttgacctaatcttattctcagacctcaagtgtctgtgcagtatctgttccatttaaatatcagctttacaattatgtggtagcctacacacataatctcatttcatcgctgtaaccaccctgttgtgataaccactattattttacccatcgtacagctgaggaagcaaacagattaagtaacttgcccaaaccagtaaatagcagacctcagactgccacccactgtccttttataatacaatttacagctatattttactttaagcaattcttttattcaaaaaccatttattaagtgcccttgcaatatcaatcgctgtgccaggcattgaatctacagatgtgagcaagacaaagtacctgtcctcaaggagctcatagtataatgaggagattaacaagaaaatgtattattacaatttagtccagtgtcatagcataaggatgatgcgaggggaaaacccgagcagtgttgccaagaggaggaaataggccaatgtggtctgggacggttggatatacttaaacatcttaataatcagagtaattttcatttacaaagagaggtcggtacttaaaataaccctgaaaaataacactggaattccttttctagcattatatttattcctgatttgcctttgccatataatctaatgcttgtttatatagtgtctggtattgtttaacagttctgtcttttctatttaaatgccactaaattttaaattcatacctttccatgattcaaaattcaaaagatcccatgggagatggttggaaaatctccacttcatcctccaagccattcaagtttcctttccagaagcaactgctactgcctttcattcatatgttcttctaaagatagtctacatttggaaatgtatgttaaaagcacgtatttttaaaatttttttcctaaatagtaacacattgtatgtctgctgtgtactttgctatttttatttattttagtgtttcttatatagcagatggaatgaatttgaagttcccagggctgaggatccatgccttctttgtttctaagttatctttcccatagcttttcattatctttcatatgatccagtatatgttaaatatgtcctacatatacatttagacaaccaccatttgttaagtatttgctctaggacagagtttggatttgtttatgtttgctcaaaaggagacccatgggctctccagggtgcactgagtcaatctagtcctaaaaagcaatcttattattaactctgtatgacagaatcatgtctggaacttttgttttctgctttctgtcaagtataaacttcactttgatgctgtacttgcaaaatcacattttctttctggaaattccggcagtgtaccttgactgctagctaccctgtgccagaaaagcctcattcgttgtgcttgaacccttgaatgccaccagctgtcatcactacacagccctcctaagaggcttcctggaggtttcgagattcagatgccctgggagatcccagagtttcctttccctcttggccatattctggtgtcaatgacaaggagtaccttggctttgccacatgtcaaggctgaagaaacagtgtctccaacagagctccttgtgttatctgtttgtacatgtgcatttgtacagtaattggtgtgacagtgttctttgtgtgaattacaggcaagaattgtggctgagcaaggcacatagtctactcagtctattcctaagtcctaactcctccttgtggtgttggatttgtaaggcactttatcccttttgtctcatgtttcatcgtaaatggcataggcagagatgatacctaattctgcatttgattgtcactttttgtacctgcattaatttaataaaatattcttatttattttgttacttggtacaccagcatgtccattttcttgtttattttgtgtttaataaaatgttcagtttaacatcccagtggagaaagttaaaaaa

(19) United States
(12) Patent Application Publication
(10) Pub. No.: US 2024/0254235 A1
(43) Pub. Date: Aug. 1, 2024

(54) COMPOSITIONS AND METHODS FOR TREATING LUNG CANCER

(71) Applicant: ASTRAZENECA AB, Södertälje (SE)

(72) Inventors: Anthony JARKOWSKI, III, Wilmington, DE (US); Phillip DENNIS, Wilmington, DE (US); Leo TRANI, Wilmington, DE (US); Michael NEWTON, Wilmington, DE (US); Norah SHIRE, Wilmington, DE (US)

(21) Appl. No.: 18/560,527

(22) PCT Filed: May 24, 2022
(86) PCT No.: PCT/EP2022/064061

§ 371 Date: Nov. 13, 2023

Related U.S. Application Data
(60) Provisional application No. 63/192,217, filed on May 24, 2021.

(51) Int. Cl. C07K 16/28; A61K 39/00; A61K 45/06; A61P 35/00

(52) U.S. Cl. CPC C07K 16/2827; A61K 45/06; A61P 35/00; A61K 2039/505; A61K 2039/545; A61K 2039/55; C07K 2317/565

(57) ABSTRACT

Disclosed are methods for treating locally advanced (Stage III), unresectable non-small-cell lung cancer (NSCLC) with an antibody that inhibits PD-1/PD-L1 activity concurrently with chemoradiation therapy (cCRT).

### BACKGROUND

Lung cancer has been the most common cancer in the world for several decades, and by 2012, there were an estimated 1.8 million new cases, representing 12.9% of all new cancers. It was also the most common cause of death from cancer, with 1.59 million deaths (19.4% of the total). Non-small cell lung cancer (NSCLC) represents approximately 80% to 85% of all lung cancers and 30% of patients present with Stage III disease. Standard treatment for patients with a good performance status (PS) and unresectable Stage III NSCLC had been platinum-based doublet chemotherapy and radiotherapy administered concurrently with curative intent (cCRT). A metanalysis of concurrent versus sequential CRT demonstrated better outcomes with concurrent therapy, but even with cCRT, 5-year overall survival (OS) ranges between 15% and 32%. As such, there remains a significant unmet need for novel therapeutic approaches to boost patient survival beyond cCRT.

Programmed cell death ligand-1 (PD-L1) on tumor and myeloid cells in the tumor microenvironment bind to the immune checkpoint protein PD-1 on activated T cells, inhibiting their activity. Durvalumab is a selective, high-affinity, human IgG1 monoclonal antibody that blocks PD-L1 binding to PD-1 and CD80, allowing T cells to recognize and kill tumor cells. Durvalumab has demonstrated encouraging antitumor activity in an early-phase clinical study across multiple advanced solid tumors, and has been approved for post-platinum, locally advanced or metastatic urothelial carcinoma.

In addressing the need for improved methods for clinical management of locally advanced cancers, the disclosure provides methods comprising administration of durvalumab concurrently with chemoradiation therapy (cCRT) to patients with late stage, locally advanced, unresectable NSCLC.

### SUMMARY

The disclosure generally relates to methods for treating locally advanced (Stage III), unresectable non-small-cell lung cancer (NSCLC) with an antibody that inhibits PD-1/PD-L1 activity concurrently with chemoradiation therapy (cCRT).

Provided herein is a method of extending progression-free survival (PFS) in a patient with unresectable non-small-cell lung cancer (NSCLC), the method comprising concurrently treating the patient with an anti-PD-L1 antibody and chemoradiation therapy.

Also provided herein is a method of increasing the overall response rate (ORR) in a patient with unresectable NSCLC, the method comprising concurrently treating the patient with an anti-PD-L1 antibody and chemoradiation therapy. Also provided herein is a combination comprising an anti-PD-L1 antibody and concurrent chemoradiation therapy for use in a method of extending progression-free survival (PFS) in a patient with unresectable non-small-cell lung cancer (NSCLC).

Also provided herein is a combination comprising an anti-PD-L1 antibody and concurrent chemoradiation therapy for use in a method of increasing the overall response rate (ORR) in a patient with unresectable non-small-cell lung cancer (NSCLC).

Also provided herein is a combination comprising an anti-PD-L1 antibody and concurrent chemoradiation therapy for use in the treatment of stage III unresectable non-small-cell lung cancer (NSCLC).

Also provided is the use of a combination comprising a human anti-PD-L1 antibody and concurrent chemoradiation therapy in the manufacture of a medicament for use in a method of extending progression-free survival (PFS) in a patient with unresectable non-small-cell lung cancer (NSCLC).

Also provided is the use of a combination comprising a human anti-PD-L1 antibody and concurrent chemoradiation therapy in the manufacture of a medicament for use in a method of increasing the overall response rate (ORR) in a patient with unresectable non-small-cell lung cancer (NSCLC).

Also provided is the use of a combination comprising a human anti-PD-L1 antibody and concurrent chemoradiation therapy in the manufacture of a medicament for use in the treatment of stage III unresectable non-small-cell lung cancer (NSCLC).

### BRIEF DESCRIPTION OF THE DRAWINGS

FIG. 1 shows the general study design for the methods disclosed herein.

FIG. 2A is a schema illustrating treatment schedule and defined endpoints. Each group contained 6 mice. Radiotherapy (RT) was delivered when tumors were approximately 100-200 mm³ and time points for assay were as indicated. FIG. 2B shows a longitudinal pathway analysis bubble diagram (from Table 4). Pathways are listed on the Y axis and time points on the X. The size of the bubble shows the absolute fold change expression values of each pathway. FIG. 2C shows tumor volumes at time of excision. Data expressed as Mean±SEM. Each group contained 6 mice. * P<0.05, Mann-Whitney test.

FIGS. 3A-3C show network maps showing the differentially regulated genes and upstream regulators between NT and RT tumors at each time point. Genes and upstream regulators are separated into their cellular compartments at day 1 (FIG. 3A), day 3 (FIG. 3B), and day 7 (FIG. 3C) post treatment with RT. FIG. 3D shows the longitudinal pathway analysis bubble diagram (from data in Table 3). Pathways are listed on the Y-axis and time points on the X. The color of the bubble indicates the direction of gene regulation. The size of the bubble shows the absolute fold change expression values of this pathway. Pathway abbreviations: Crosstalk between dendritic cells (DC) and natural killer (NK) cells; Recognition of bacteria and viruses—Role of pattern recognition receptors in recognition of bacteria and viruses; Innate and adaptive immune system—Communication between innate and adaptive immune cells.

FIGS. 4A-4F show that RT leads to changes in tumor-infiltrating myeloid cell populations. Cells were isolated from non-treated (NT) (black bars) or RT-treated (7 Gy) (grey bars) tumors 1, 3, or 7 days post RT (or time-matched NT controls). FIG. 4A shows tumor samples analyzed by flow cytometry for the presence of F4/80⁺ cells. FIGS. 4B and 4C show expression (MFI) of CD86 and CD206 on F4/80⁺ cells. Representative histograms are shown above corresponding bar charts with isotype control (black line), NT (black filled), and RT (grey line). FIG. 4D shows the percentages of CD86⁺ and CD206⁺ cells that were F4/80⁺, and box-whisker plots show the CD86⁺/CD206⁺ ratio for NT (black) or RT-treated (grey) tumors.

FIGS. 4E and 4F show the frequencies of CD11b⁺Gr1^{lo} and CD11b⁺Gr1^{hi} cells in the tumor tissue. Plotted are means±SEM. Each group contained 6 mice. * P<0.05 and ** P<0.01 when comparing NT with irradiated groups.

FIGS. 5A-5F show that RT impacted the frequency and phenotype of tumor-infiltrating lymphocytes. Cells were isolated from non-treated (NT) (black bars) or RT-treated (7 Gy) (grey bars) tumors 1, 3, or 7 days post RT (or time-matched NT controls). FIGS. 5A and 5B show the frequency of CD4⁺ and CD8⁺ tumor-infiltrating T-cells. FIGS. 5C and 5D show the CD69 expression on CD4⁺ and CD8⁺ T-cells. FIG. 5E shows tumor-infiltrating CD4⁺CD25⁺FoxP3⁺ (T_{reg}) cells expressed as a percentage of CD4⁺ population. FIG. 5F shows the ratio of CD8⁺ to T_{reg} cells. Plotted are means±SEM. Each group contained 6 mice. *P<0.05 and ** P<0.01 when comparing NT with irradiated groups.

FIGS. 6A-6G show that RT resulted in elevated expression of both PD-1 and PD-L1 in the tumor, which attenuated the efficacy of treatment. Cells were isolated from non-treated (NT) (black bars) or RT-treated (7 Gy) (grey bars) tumors 1, 3, or 7 days post RT (or time-matched NT controls). FIGS. 6A and 6B show PD-1 expression on CD4⁺ and CD8⁺ T-cells. FIGS. 6C and 6D show PD-L1 expression on CD4⁺ and CD8⁺ T-cells. FIG. 6E show PD-L1 expression on CD45⁻ tumor cells. Plotted are means±SEM. * P<0.05 and ** P<0.01 when comparing NT with irradiated groups. FIGS. 6F and 6G show tumor growth curves and Kaplan-Meier curve of mice bearing established tumor following treatment with 7 Gy RT alone or in combination with αPD-L1 mAb dosed at 10 mg/kg 3qw for 1 week. Experimental groups contained at least 6 mice and are representative of 2 independent studies. ⁺⁺ P<0.01 relative to 7 Gy RT alone. ** P<0.01 and *** P<0.001 relative to NT control.

FIGS. 7A-7B show heat maps showing the fold-changes in lineage (FIG. 7A) and phenotype (FIG. 7B) markers from irradiated tumor tissue removed day 1, 3, and 7 following 7 Gy RT, as a percentage of time-matched untreated control tumors. Bold boxed values are those which were statistically significant relative to untreated time-matched samples. Each group contained 6 mice. (Mann Whitney U, P<0.05).

FIGS. 8A-8D show gating strategies employed for analysis of tumor cell populations. FIG. 8A shows gating on live leukocytes isolated from the spleen used to draw the leukocyte gate. FIG. 8B shows CD4⁺ CD25⁺ FoxP3⁺ cells in tumor tissue. FIG. 8C shows CD11b⁺Gr1^{lo} and ^{hi} populations in spleen and tumor tissue. FIG. 8D shows CD45⁻ tumor cells.

FIG. 9 shows the percentage of CD45⁺ cells in tumor tissue isolated either 1, 3, or 7 days after NT (black) or 7 Gy (grey) RT. P<0.01, Mann Whitney U. Each group contained 6 mice. ** P<0.01, Mann-Whitney test.

FIGS. 10A-10B show representative histograms of CD69 (FIG. 10A) and PD-1 (FIG. 10B) expression in CD4⁺ and CD8⁺ cells from NT and RT-treated tumor tissue. FIG. 10A shows the unfilled histograms are isotype controls. FIG. 10B shows patterned histogram are isotype control, black line is NT, grey line is 7 Gy RT.

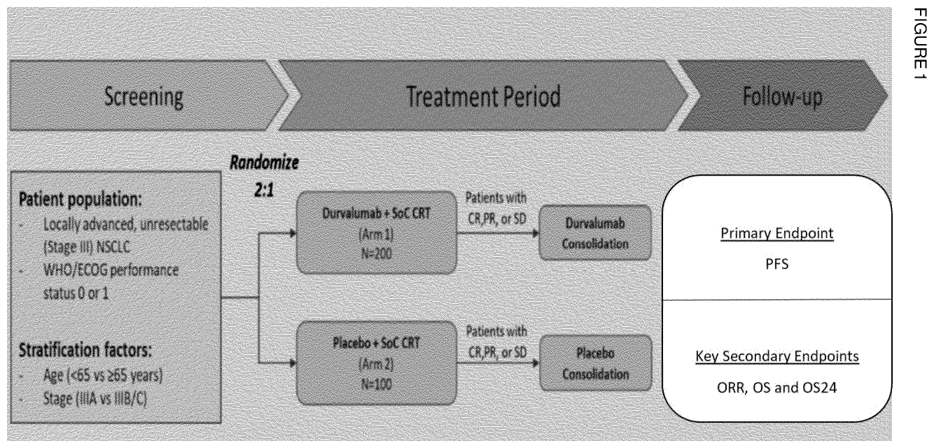

### DETAILED DESCRIPTION

Unless defined otherwise, all technical and scientific terms used herein have the meaning commonly understood by a person skilled in the art to which this disclosure belongs. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

In this disclosure, “comprises,” “comprising,” “containing” and “having” and the like can have the meaning ascribed to them in U.S. patent law and can mean “includes,” “including,” and the like; “consisting essentially of” or “consists essentially” likewise have the meaning ascribed in U.S. patent law and are open-ended, allowing for the presence of more than that which is recited so long as basic or novel characteristics of that which is recited are not changed by the presence of more than that which is recited, but excludes prior art aspects.

Unless specifically stated or obvious from context, the term “or,” as used herein, is understood to be inclusive. Unless specifically stated or obvious from context, the terms “a,” “an,” and “the,” as used herein, are understood to be singular or plural.

Unless specifically stated or obvious from context, the term “about,” as used herein, is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Any compositions or methods provided herein can be combined with one or more of any of the other compositions and methods provided herein.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

“Anti-PD-L1 antibody,” as used herein, refers to an antibody or antigen-binding fragment thereof that selectively binds a PD-L1 polypeptide. Exemplary anti-PD-L1 antibodies are described, for example, in U.S. Pat. Nos. 8,779,108 and 9,493,565, which are incorporated herein by reference.

The term “durvalumab,” as used herein, refers to an antibody that selectively binds PD-L1 and blocks the binding of PD-L1 to PD-1 and CD80 receptors, as disclosed in U.S. Pat. No. 9,493,565 (wherein durvalumab is referred to as “2.14H9OPT”), which is incorporated by reference herein in its entirety. The fragment crystallizable (Fc) domain of durvalumab contains a triple mutation in the constant domain of the IgG1 heavy chain that reduces binding to the complement component C1q and the Fcγ receptors responsible for mediating antibody-dependent cell-mediated cytotoxicity (“ADCC”). Durvalumab can relieve PD-L1-mediated suppression of human T-cell activation in vitro and inhibits tumor growth in a xenograft model via a T-cell dependent mechanism.

A “complete response” (CR) refers to the disappearance of all lesions, whether measurable or not, and no new lesions. Confirmation can be obtained using a repeat, consecutive assessment no less than four weeks from the date of first documentation. New, non-measurable lesions preclude CR.

A “partial response” (PR) refers to a decrease in tumor burden ≥50% relative to baseline. Confirmation can be obtained using a consecutive repeat assessment at least four weeks from the date of first documentation.

“Progressive disease” (PD) refers to an increase in tumor burden ≥25% relative to the minimum recorded (nadir). Confirmation can be obtained by a consecutive repeat assessment at least four weeks from the date of first documentation. New, non-measurable lesions do not define PD.

“Stable disease” (SD) refers to not meeting the criteria for CR, PR, or PD. SD indicates a decrease in tumor burden of 50% relative to baseline cannot be established and a 25% increase compared to nadir cannot be established.

Non-small cell lung cancer (NSCLC) can refer to any of the three main subtypes of NSCLC: squamous cell carcinoma, adenocarcinoma, and large cell (undifferentiated) carcinoma. Other subtypes include adenosquamous carcinoma and sarcomatoid carcinoma.

As used herein, “PD-L1” may refer to polypeptide or polynucleotide sequences, or fragments thereof, having at least about 85%, 95%, or 100% sequence identity to PD-L1 sequences. PD-L1 is also referred to in the art as B7-H1. In some embodiments, the PD-L1 polypeptide, or fragment thereof, has at least about 85%, 95%, or 100% sequence identity to NCBI Accession No. NP_001254635, and has PD-1 and CD80 binding activity.

### PD-L1 Polypeptide Sequence

| NCBI ACCESSION NO. NP_001254635 | |
|---|---|
| 1 | mrifavfifm tywhllnapy nkinqrilvv dpvtsehelt cqaegypkae viwtssdhqv |
| 61 | lsgkttttns kreeklfnvt stlrintttn eifyctfrrl dpeenhtael vipelplahp |
| 121 | pnerthlvil gaillclgva ltfifrlrkg rmmdvkkcgi qdtnskkqsd thleet |

In some embodiments, a “PD-L1 nucleic acid molecule” comprises a polynucleotide encoding a PD-L1 polypeptide. An exemplary PD-L1 nucleic acid molecule sequence is provided in NCBI Accession No. NM_001267706.

### PD-L1 Nucleic Acid Sequence

| NCBI ACCESSION NO. NM_001267706 mRNA | |
|---|---|
| 1 | ggcgcaacgc tgagcagctg gcgcgtcccg cgcggcccca gttctgcgca gcttcccgag |
| 61 | gctccgcacc agccgcgctt ctgtccgcct gcagggcatt ccagaaagat gaggatattt |
| 121 | gctgtcttta tattcatgac ctactggcat ttgctgaacg ccccatacaa caaaatcaac |
| 181 | caaagaattt tggttgtgga tccagtcacc tctgaacatg aactgacatg tcaggctgag |
| 241 | ggctacccca aggccgaagt catctggaca agcagtgacc atcaagtcct gagtggtaag |
| 301 | accaccacca ccaattccaa gagagaggag aagcttttca atgtgaccag cacactgaga |
| 361 | atcaacacaa caactaatga gattttctac tgcactttta ggagattaga tcctgaggaa |
| 421 | aaccatacag ctgaattggt catcccagaa ctacctctgg cacatcctcc aaatgaaagg |
| 481 | actcacttgg taattctggg agccatctta ttatgccttg gtgtagcact gacattcatc |
| 541 | ttccgtttaa gaaaagggag aatgatggat gtgaaaaaat gtggcatcca agatacaaac |
| 601 | tcaaagaagc aaagtgatac acatttggag gagacgtaat ccagcattgg aacttctgat |
| 661 | cttcaagcag ggattctcaa cctgtggttt aggggttcat cggggctgag cgtgacaaga |
| 721 | ggaaggaatg ggcccgtggg atgcaggcaa tgtgggactt aaaaggccca agcactgaaa |
| 781 | atggaacctg gcgaaagcag aggaggagaa tgaagaaaga tggagtcaaa cagggagcct |
| 841 | ggagggagac cttgatactt tcaaatgcct gaggggctca tcgacgcctg tgacagggag |
| 901 | aaaggatact tctgaacaag gagcctccaa gcaaatcatc cattgctcat cctaggaaga |
| 961 | cgggttgaga atccctaatt tgagggtcag ttcctgcaga agtgcccttt gcctccactc |
| 1021 | aatgcctcaa tttgttttct gcatgactga gagtctcagt gttggaacgg gacagtattt |
| 1081 | atgtatgagt ttttcctatt tattttgagt ctgtgaggtc ttcttgtcat gtgagtgtgg |
| 1141 | ttgtgaatga tttcttttga agatatattg tagtagatgt tacaattttg tcgccaaact |
| 1201 | aaacttgctg cttaatgatt tgctcacatc tagtaaaaca tggagtattt gtaaggtgct |
| 1261 | tggtctcctc tataactaca agtatacatt ggaagcataa agatcaaacc gttggttgca |
| 1321 | taggatgtca cctttattta acccattaat actctggttg acctaatctt attctcagac |
| 1381 | ctcaagtgtc tgtgcagtat ctgttccatt taaatatcag ctttacaatt atgtggtagc |
| 1441 | ctacacacat aatctcattt catcgctgta accaccctgt tgtgataacc actattattt |
| 1501 | tacccatcgt acagctgagg aagcaaacag attaagtaac ttgcccaaac cagtaaatag |
| 1561 | cagacctcag actgccaccc actgtccttt tataatacaa tttacagcta tattttactt |
| 1621 | taagcaattc ttttattcaa aaaccattta ttaagtgccc ttgcaatatc aatcgctgtg |
| 1681 | ccaggcattg aatctacaga tgtgagcaag acaaagtacc tgtcctcaag gagctcatag |
| 1741 | tataatgagg agattaacaa gaaaatgtat tattacaatt tagtccagtg tcatagcata |
| 1801 | aggatgatgc gaggggaaaa cccgagcagt gttgccaaga ggaggaaata ggccaatgtg |
| 1861 | gtctgggacg gttggatata cttaaacatc ttaataatca gagtaatttt catttacaaa |
| 1921 | gagaggtcgg tacttaaaat aaccctgaaa aataacactg gaattccttt tctagcatta |
| 1981 | tatttattcc tgatttgcct ttgccatata atctaatgct tgtttatata gtgtctggta |
| 2041 | ttgtttaaca gttctgtctt ttctatttaa atgccactaa attttaaatt catacctttc |
| 2101 | catgattcaa aattcaaaag atcccatggg agatggttgg aaaatctcca cttcatcctc |
| 2161 | caagccattc aagtttcctt tccagaagca actgctactg cctttcattc atatgttctt |
| 2221 | ctaaagatag tctacatttg gaaatgtatg ttaaaagcac gtatttttaa aatttttttc |
| 2281 | ctaaatagta acacattgta tgtctgctgt gtactttgct atttttattt attttagtgt |
| 2341 | ttcttatata gcagatggaa tgaatttgaa gttcccaggg ctgaggatcc atgccttctt |
| 2401 | tgtttctaag ttatctttcc catagctttt cattatcttt catatgatcc agtatatgtt |
| 2461 | aaatatgtcc tacatataca tttagacaac caccatttgt taagtatttg ctctaggaca |
| 2521 | gagtttggat ttgtttatgt ttgctcaaaa ggagacccat gggctctcca gggtgcactg |
| 2581 | agtcaatcta gtcctaaaaa gcaatcttat tattaactct gtatgacaga atcatgtctg |
| 2641 | gaacttttgt tttctgcttt ctgtcaagta taaacttcac tttgatgctg tacttgcaaa |
| 2701 | atcacatttt ctttctggaa attccggcag tgtaccttga ctgctagcta ccctgtgcca |
| 2761 | gaaaagcctc attcgttgtg cttgaaccct tgaatgccac cagctgtcat cactacacag |
| 2821 | ccctcctaag aggcttcctg gaggtttcga gattcagatg ccctgggaga tcccagagtt |
| 2881 | tcctttccct cttggccata ttctggtgtc aatgacaagg agtaccttgg ctttgccaca |
| 2941 | tgtcaaggct gaagaaacag tgtctccaac agagctcctt gtgttatctg tttgtacatg |
| 3001 | tgcatttgta cagtaattgg tgtgacagtg ttctttgtgt gaattacagg caagaattgt |
| 3061 | ggctgagcaa ggcacatagt ctactcagtc tattcctaag tcctaactcc tccttgtggt |
| 3121 | gttggatttg taaggcactt tatccctttt gtctcatgtt tcatcgtaaa tggcataggc |
| 3181 | agagatgata cctaattctg catttgattg tcactttttg tacctgcatt aatttaataa |
| 3241 | aatattctta tttattttgt tacttggtac accagcatgt ccattttctt gtttattttg |
| 3301 | tgtttaataa aatgttcagt ttaacatccc agtggagaaa gttaaaaaa |

Programmed Death-1 (“PD-1”) is an approximately 31 kD type I membrane protein member of the extended CD28/CTLA4 family of T cell regulators (see Ishida et al., “Induced Expression of PD-1, A Novel Member of the Immunoglobulin Gene Superfamily, Upon Programmed Cell Death,” EMBO J. 11: 3887-95 (1992)).

PD-1 is expressed on activated T cells, B cells, and monocytes (Agata et al., “Expression of the PD-1 Antigen on the Surface of Stimulated Mouse T and B Lymphocytes,” Int. Immunol. 8(5): 765-72 (1996); Yamazaki et al., “Expression of Programmed Death 1 Ligands by Murine T Cells and APC,” J. Immunol. 169: 5538-45 (2002)) and at low levels in natural killer (NK) T cells (Nishimura et al., “Facilitation of Beta Selection and Modification of Positive Selection in the Thymus of PD-l-Deficient Mice,” J. Exp. Med. 191: 891-98 (2000); Martin-Orozco et al., “Inhibitory Costimulation and Anti-Tumor Immunity,” Semin. Cancer Biol. 17(4): 288-98 (2007)). PD-1 is a receptor responsible for down-regulation of the immune system following activation by binding of PDL-1 or PDL-2 (Martin-Orozco et al. (2007)) and functions as a cell death inducer (Ishida et al. (1992); Subudhi et al., “The Balance of Immune Responses: Costimulation Verse Coinhibition,” J. Molec. Med. 83: 193-202 (2005); Lazar-Molnar et al., “Crystal Structure of the Complex Between Programmed Death-1 (PD-1) and Its Ligand PD-L2,” Proc. Natl. Acad. Sci. U.S.A. 105(30): 10483-88 (2008)). This process is exploited in many tumours via the over-expression of PD-L1, leading to a suppressed immune response.

PD-1 is a well-validated target for immune mediated therapy in oncology, with positive clinical trials in the treatment of melanoma and non-small cell lung cancers (NSCLC), among others. Antagonistic inhibition of the PD-1/PDL-1 interaction increases T cell activation, enhancing recognition and elimination of tumour cells by the host immune system. The use of anti-PD-1 antibodies to treat infections and tumors and up-modulate an adaptive immune response has been proposed.

The term “antibody,” as used herein, refers to an immunoglobulin or a fragment or a derivative thereof, and encompasses any polypeptide comprising an antigen-binding site, regardless whether it is produced in vitro or in vivo. The term includes, but is not limited to, polyclonal, monoclonal, monospecific, polyspecific, non-specific, humanized, human single-chain, chimeric, synthetic, recombinant, hybrid, mutated, and grafted antibodies. Unless otherwise modified by the term “intact,” as in “intact antibodies,” for the purposes of this disclosure, the term “antibody” also includes antibody fragments such as Fab, F(ab′)₂, Fv, scFv, Fd, dAb, and other antibody fragments that retain antigen-binding function, i.e., the ability to bind PD-L1 specifically. Typically, such fragments would comprise an antigen-binding domain.

The term “human antibody,” as used herein, includes antibodies having variable and constant regions substantially corresponding to human germline immunoglobulin sequences.

The terms “antigen-binding domain,” “antigen-binding fragment,” and “binding fragment,” as used herein, refer to a part of an antibody molecule that comprises amino acids responsible for the specific binding between the antibody and the antigen. In some instances, where an antigen is large, the antigen-binding domain may only bind to a part of the antigen. A portion of the antigen molecule that is responsible for specific interactions with the antigen-binding domain is referred to as “epitope” or “antigenic determinant.” An antigen-binding domain typically comprises an antibody light chain variable region (V_{L}) and an antibody heavy chain variable region (V_{H}); however, it does not necessarily have to comprise both. For example, a so-called Fd antibody fragment consists only of a V_{H} domain, but still retains some antigen-binding function of the intact antibody.

Binding fragments of an antibody are produced by recombinant DNA techniques, or by enzymatic or chemical cleavage of intact antibodies. Binding fragments include Fab, Fab′, F(ab′)2, Fv, and single-chain antibodies. An antibody other than a “bispecific” or “bifunctional” antibody is understood as an antibody in which each of its binding sites is identical. Digestion of antibodies with the enzyme papain results in two identical antigen-binding fragments, known also as “Fab” fragments, and a “Fc” fragment, having no antigen-binding activity but having the ability to crystallize. Digestion of antibodies with the enzyme pepsin results in a F(ab′)2 fragment in which the two arms of the antibody molecule remain linked and comprise two-antigen binding sites. The F(ab′)2 fragment has the ability to crosslink antigen. The term “Fv,” as used herein, refers to the minimum fragment of an antibody that retains both antigen-recognition and antigen-binding sites. The term “Fab,” as used herein, refers to a fragment of an antibody that comprises the constant domain of the light chain and the CHI domain of the heavy chain.

The term “mAb,” as used herein, refers to a monoclonal antibody. Antibodies of the disclosure comprise, without limitation, whole native antibodies, bispecific antibodies, chimeric antibodies, Fab, Fab′, single chain V region fragments (scFv), fusion polypeptides, and unconventional antibodies.

The terms “isolated,” “purified,” or “biologically pure,” as used herein, refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences.

The term “specifically binds,” as used herein, is meant to refer to a compound (e.g., an antibody) that recognizes and binds a molecule (e.g., a polypeptide), but that does not substantially recognize and bind other molecules in a sample, for example, a biological sample. For example, two molecules that specifically bind form a complex that is relatively stable under physiologic conditions. Specific binding is characterized by a high affinity and a low to moderate capacity as distinguished from nonspecific binding which usually has a low affinity with a moderate to high capacity. Typically, binding is considered specific when the affinity constant K_{A} is higher than 10⁶ M⁻¹, or more preferably higher than 10⁸ M⁻¹. If necessary, non-specific binding can be reduced without substantially affecting specific binding by varying the binding conditions. The appropriate binding conditions, such as concentration of antibodies, ionic strength of the solution, temperature, time allowed for binding, concentration of a blocking agent (e.g., serum albumin, milk casein), may be optimized by a skilled artisan using routine techniques.

As generally used herein, the terms “treat,” “treating,” “treatment,” and the like, refer to reducing, ameliorating, or slowing the progression of a disorder or disease and/or symptoms associated with a disorder or disease. It will be appreciated that, although not precluded, treating a disorder, disease, or condition does not require that the disorder, disease, or condition or associated symptoms be completely eliminated. In particular embodiments relating to NSCLC, “treat,” “treating,” “treatment,” can refer to achieving any one or combination of primary or secondary clinical endpoints.

Provided herein is a method of extending progression-free survival (PFS) in a patient with unresectable non-small-cell lung cancer (NSCLC), the method comprising concurrently treating the patient with a human anti-PD-L1 antibody and chemoradiation therapy.

Also provided herein is a method of increasing the overall response rate (ORR) in a patient with unresectable NSCLC, the method comprising concurrently treating the patient with a human anti-PD-L1 antibody and chemoradiation therapy.

Also provided herein is a method treating a patient with stage III unresectable NSCLC, the method comprising concurrently treating the patient with a human anti-PD-L1 antibody and chemoradiation therapy.

In some embodiments, the human anti-PD-L1 antibody is durvalumab, avelumab, atezolizumab or sugemalimab. In some embodiments, the human anti-PD-L1 antibody is durvalumab, avelumab, or atezolizumab. In some embodiments, the human anti-PD-L1 antibody is durvalumab.

Durvalumab and antigen-binding fragments thereof for use in the methods provided herein comprises a heavy chain and a light chain or a heavy chain variable region and a light chain variable region. The amino acid sequence of the durvalumab light chain variable region is provided in SEQ ID NO: 1, and the amino acid sequence of the durvalumab heavy chain variable region is provided in SEQ ID NO: 2. The amino acid sequences of the durvalumab heavy chain variable region complementarity determining regions (CDRs) are provided in SEQ ID NO: 3 (CDR1), SEQ ID NO: 4 (CDR2), and SEQ ID NO: 5 (CDR3), and the amino acid sequences of the durvalumab light chain variable region CDRs are provided in SEQ ID NO: 6 (CDR1), SEQ ID NO: 7 (CDR2), and SEQ ID NO: 8 (CDR3).

In some embodiments, durvalumab or an antigen-binding fragment thereof for use in the methods provided herein comprises a light chain variable region comprising the amino acid sequence of SEQ ID NO: 1 and a heavy chain variable region comprising the amino acid sequence of SEQ ID NO: 2. In some embodiments, durvalumab or an antigen-binding fragment thereof for use in the methods provided herein comprises a heavy chain variable region and a light chain variable region, wherein the heavy chain variable region comprises the Kabat-defined CDR1, CDR2, and CDR3 sequences of SEQ ID NOs: 3-5, and wherein the light chain variable region comprises the Kabat-defined CDR1, CDR2, and CDR3 sequences of SEQ ID NOs: 6-8. Those of ordinary skill in the art would easily be able to identify Chothia-defined, Abm-defined or other CDR definitions known to those of ordinary skill in the art. In some embodiments, durvalumab or an antigen-binding fragment thereof for use in the methods provided herein comprises the variable heavy chain and variable light chain CDR sequences of the 2.14H9OPT antibody as disclosed in U.S. Pat. Nos. 8,779,108 and 9,493,565, which are herein incorporated by reference in their entirety.

Durvalumab or an antigen-binding fragment thereof can be administered once every four weeks while providing benefit to the patient. In further embodiments, the patient is administered additional follow-on doses. Follow-on doses can be administered at various time intervals depending on the patient's age, weight, clinical assessment, tumor burden, and/or other factors, including the judgment of the attending physician.

In some embodiments, multiple doses of durvalumab or an antigen-binding fragment thereof are administered to the patient. In some embodiments, at least three doses, at least four doses, at least five doses, at least six doses, at least seven doses, at least eight doses, at least nine doses, at least ten doses, at least fifteen doses, at least twenty-six doses, or more than at least twenty doses can be administered to the patient. In some embodiments, durvalumab or an antigen-binding fragment thereof is administered every two weeks, over a two week period, over a four-week treatment period, over a six-week treatment period, over an eight-week treatment period, over a twelve-week treatment period, over a twenty-four-week treatment period, over a one-year treatment period, or more than over a one-year treatment period.

In some embodiments, the interval between doses can be every three weeks. In some embodiments, the interval between doses can be every four weeks (Q4W). In some embodiments, the intervals between doses can be every two months (e.g., during a maintenance phase).

In some embodiments, the patient is administered one or more doses of the anti-PD-L1 or an antigen-binding fragment thereof, wherein the dose is a fixed dose of 1500 mg. In some embodiments, the patient is administered 1500 mg of the human anti-PD-L1 every four weeks. In some embodiments, the patient is administered one or more doses of the anti-PD-L1 wherein the dose is about 20 mg/kg. In some embodiments, the patient is administered 1500 mg of the human anti-PD-L1 antibody, intravenously, every four weeks (Q4W).

In some embodiments, the patient is administered one or more doses of durvalumab or an antigen-binding fragment thereof, wherein the dose is a fixed dose of 1500 mg. In some embodiments, the patient is administered 1500 mg of durvalumab every four weeks. In some embodiments, the patient is administered one or more doses of durvalumab wherein the dose is about 20 mg/kg.

The amount of durvalumab or an antigen-binding fragment thereof to be administered to the patient may be adjusted and can depend on various parameters, such as the patient's age, weight, clinical assessment, tumor burden and/or other factors, including the judgment of the attending physician. In some embodiments, the dose is a fixed dose.

In some embodiments, administration of durvalumab or an antigen-binding fragment thereof according to the methods provided herein is through parenteral administration. For example, durvalumab or an antigen-binding fragment thereof can be administered by intravenous infusion or by subcutaneous injection. In some embodiments, the administration is by intravenous infusion.

In some embodiments, durvalumab or an antigen-binding fragment thereof is administered concurrently with chemoradiation therapy. The term “concurrently,” as used herein, refers to the administration of durvalumab or an antigen-binding fragment thereof and administration of chemoradiation therapy within about three days of each other. In some embodiments, durvalumab or an antigen-binding fragment thereof is administered within about two days of chemoradiation therapy. In some embodiments, durvalumab or an antigen-binding fragment thereof is administered within about one day of chemoradiation therapy. In some embodiments, durvalumab or an antigen-binding fragment thereof is administered on Cycle 1 Day 1 of chemoradiation therapy.

In some embodiments, the anti-PD-L1 antibody is administered on the first day of chemoradiation therapy.

In some embodiments, chemoradiation therapy comprises a platinum-based therapeutic agent.

In some embodiments, the concurrent chemoradiation therapy comprises any accepted standard first-line treatments for patients with advanced NSCLC. In some embodiments, standard first-line treatments may include chemotherapy, radiation therapy, or both (chemoradiation therapy). In some embodiments, the therapy can comprise one or more platinum-based chemotherapeutic agents. In some embodiments, the chemoradiation therapy is platinum-based. In some embodiments, the one or more platinum-based chemotherapeutic agents can be selected from carboplatin, cisplatin, oxaliplatin, or combinations thereof. As described herein, the platinum-based therapy can comprise singlet or doublet regimens such as, for example, administering cisplatin or carboplatin with another anticancer agent such as paclitaxel, docetaxel, etoposide, gemcitabine, vinorelbine, and the like.

The disclosure relates to methods of treating patients who have unresectable locally advanced non-small-cell lung cancer (NSCLC), comprising concurrently administering to the patient a human anti-PD-L1 antibody and chemoradiation therapy. The disclosed methods of treatment can provide for substantial improvement in a patient's progression-free survival (PFS), overall response rate (ORR), overall survival (OS), and proportion of patients alive at 24 months from randomization (OS24).

In some embodiments, the method provides an increase in PFS relative to placebo. In some embodiments, the method provides an increase in ORR relative to placebo. In some embodiments, the method provides an increase in OS versus placebo.

In some embodiments, there is provided a combination comprising a human anti-PD-L1 antibody and concurrent chemoradiation therapy for use in a method of extending progression-free survival (PFS) in a patient with unresectable non-small-cell lung cancer (NSCLC). In some embodiments, there is provided a combination comprising a human anti-PD-L1 antibody and concurrent chemoradiation therapy for use in a method of increasing the overall response rate (ORR) in a patient with unresectable non-small-cell lung cancer (NSCLC). In some embodiments, there is provided a combination comprising a human anti-PD-L1 antibody and concurrent chemoradiation therapy for use in the treatment of stage III unresectable non-small-cell lung cancer (NSCLC).

In some embodiments, there is provided the use of a combination comprising a human anti-PD-L1 antibody and concurrent chemoradiation therapy in the manufacture of a medicament for use in a method of extending progression-free survival (PFS) in a patient with unresectable non-small-cell lung cancer (NSCLC). In some embodiments, there is provided the use of a combination comprising a human anti-PD-L1 antibody and concurrent chemoradiation therapy in the manufacture of a medicament for use in a method of increasing the overall response rate (ORR) in a patient with unresectable non-small-cell lung cancer (NSCLC). In some embodiments, there is provided the use of a combination comprising a human anti-PD-L1 antibody and concurrent chemoradiation therapy in the manufacture of a medicament for use in the treatment of stage III unresectable non-small-cell lung cancer (NSCLC).

Overall Survival (OS) relates to the time period beginning on the date of treatment until death due to any cause. OS may refer to overall survival within a period of time such as, for example, 12 months, 18 months, 24 months, and the like. Such periods of time can be identified, for example, as “OS24” which refers to the number (%) of patients who are alive at 24 months after treatment onset per the Kaplan-Meier estimate of overall survival at 24 months.

Progression-Free Survival (PFS) relates to the time period beginning on the date of treatment until the date of objective disease progression (RECIST 1.1) or death (by any cause in the absence of progression). In some embodiments, the methods of the disclosure provide for increase in PFS. In some embodiments, the methods provide for PFS of at least 9 months to at least about 24 months (e.g., at least 9, 10, 11, 12, 13, 14, 15, 16, 17, 18, 19, 20, 21, 22, 23, 24, or more than 24 months, and up to about 5 years).

Objective Response Rate (ORR) refers to the number (%) of patients with at least one visit response of Complete Response (CR) or partial response (PR) per RECIST 1.1.

As described herein, and illustrated by the Examples, the methods provide for the treatment of locally advanced, unresectable NSCLC. In some embodiments, an unresectable cancer includes cancer that cannot be removed completely through surgery for at least one of several medical reasons. Reasons why a cancer may be unresectable include, for example, tumor size (e.g., too large to safely remove and/or may require extensive removal of a part of an essential organ), tumor location (e.g., tumor physically intertwined with vital structures such as blood vessels or nerves), tumor metastasis where removal of the tumor will not be effective to control all of the cancer, or other medical conditions that heighten risk of surgery to an unacceptable level (e.g., heart disease, lung disease, diabetes). Further, an unresectable NSCLC may not be permanently unresectable after aggressive treatment that may be effective to reduce the size of a tumor to a degree that allows for possible surgical resection. Further, unresectable NSCLC can also refer to NSCLC (or remote metastases) that will not be completely removed by surgery, but which may be partially removed by one or more surgical procedures. Examples include debulking surgery and surgery that removes parts of the lung cancer as well as parts of metastatic lesions.

In certain embodiments, the methods disclosed herein can be used on resectable cancers.

As described and illustrated herein, the methods of the disclosure can be used for treatment of patients with late-stage (e.g., Stage III) locally advanced, unresectable NSCLC. Cancer staging can be performed using any tests that are generally known and accepted in the art. In some embodiments, the cancer staging can comprise the American Joint Committee on Cancer's (AJCC's) TNM system. Generally, the TNM system provides results from various tests and scans in order to determine the size and location of the primary tumor (Tumor, T); whether the cancer has spread to the lymph nodes, and if it has, the location and number of the affected lymph nodes (Node, N); and whether the cancer has spread to other parts of the body, and if it has, the extent and location of the remote cancer (Metastasis, M). While each type of cancer may have its own specific system, the TNM staging system generally uses scaled scoring for each letter.

In some embodiments, the unresectable NSCLC is stage III. In some embodiments, the unresectable NSCLC is locally advanced. In some embodiments, the unresectable NSCLC is stage III and locally advanced.

For Tumor, “T” is associated with a number (e.g., 0 to 4) to describe the general tumor size, location, and whether it intrudes into nearby tissues. Larger or more intrusive tumors are given a higher number and, depending on the cancer, a lowercase letter, such as “a,” “b,” or “m” (for multiple), may be added to provide more detail.

Similarly, for Node, “N” is associated with a number (e.g., 0 to 3) to describe whether cancer has been found in the lymph nodes, and can also indicate the number of lymph nodes containing cancer. Larger numbers are assigned when more lymph nodes are involved with cancer.

For Metastasis, “M” indicates whether or not the cancer has spread to other parts of the body and is labeled M0 for no spread, or M1 if it has spread.

The T, N, and M results are combined to determine the stage of cancer, typically one of four stages: stages I (one) to IV (four). Some cancers also have a stage 0 (zero). Stage 0 describes cancer in situ, remaining local to the original tissue without any spread to nearby tissues. This stage of cancer is often highly curable, usually by removing the entire tumor with surgery. Stage I or early-stage cancer, is typically used to describe a small cancer or tumor that has not grown deeply into nearby tissues, and has not spread to the lymph nodes or other parts of the body. Stage II and III describe larger cancers or tumors that have grown more deeply into nearby tissue, and that may have also spread to lymph nodes but not metastasized to other tissues. Stage IV describes a cancer that has spread to other organs or parts of the body and often identified as advanced or metastatic cancer.

Staging may include optional analysis of prognostic factors to provide chances of recovery and a recommended therapy. Prognostic factors may include grading the cancer based on appearance of the cancer cells; analysis of tumor marker expression; and analysis of tumor genetics.

A cancer may be restaged using the same initial system in order to determine efficacy of a treatment or obtain more information about a recurrent cancer.

Staging of NSCLC: NSCLC has 5 stages: a stage 0 (zero) and stages I through IV (1 through 4). Stage 0 NSCLC indicates that the cancer has not grown into nearby tissues or spread outside the lung.

Stage I NSCLC indicates that the cancer is a small tumor that has not spread to any lymph nodes. Stage I is divided into 2 sub-stages based on the size of the tumor: Stage IA tumors are less than 3 centimeters (cm) wide, and Stage IB tumors are more than 3 cm but less than 5 cm wide. Stage I NSCLC may allow for complete surgical removal of the cancer.

Stage II is divided into 2 sub-stages (IIA and IIB). Stage IIA can be either a tumor larger than 5 cm but less than 7 cm wide that has not spread to the nearby lymph nodes, or a small tumor less than 5 cm wide that has spread to the nearby lymph nodes. Stage IIB can describe either a tumor larger than 5 cm but less than 7 cm wide that has spread to the lymph nodes, or a tumor more than 7 cm wide that may or may not have grown into nearby structures in the lung but has not spread to the lymph nodes. While stage II NSCLC may allow for surgical treatment, other therapies are commonly required to treat this stage of NSCLC.

Stage III includes sub-stages IIIA or IIIB. Surgery is difficult or impossible in many stage IIIA cancers and nearly all stage IIIB cancers, because of the spread of the cancer to the lymph nodes or because of its growth into nearby structures in the lung. Surgery in either situation typically requires the partial removal of the cancer.

Stage IV NSCLC is associated with the spread to more than one area in the other lung, the fluid surrounding the lung or the heart, or distant metastasis in the body. NSCLC is more likely to spread to the brain, bones, liver, and adrenal glands. Stage IV NSCLC includes substages IVA (spread within the chest) and IVB (spread outside of the chest). Surgery is rarely successful for most stage III or IV NSCLC and may be impossible to remove if it has spread to the lymph nodes above the collarbone, or to vital structures within the chest (e.g., heart, large blood vessels, or the main pulmonary structures). In certain embodiments, a patient disclosed herein is a stage IV NSCLC patient.

Recurrent NSCLC is detected after a course of treatment.

The practice of the methods disclosed herein employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology”; “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); and “Current Protocols in Immunology” (Coligan, 1991).

### EXAMPLES

### Example 1: Efficacy of Durvalumab in Combination with Platinum-Based Chemoradiation Therapy in Patients with Locally Advanced, Unresectable Non-Small Cell Lung Cancer (Stage III)

This is a Phase III, randomized, double-blind, placebo-controlled, multi-center, international study assessing the efficacy and safety of durvalumab given concurrently with platinum-based chemoradiation therapy (CRT) (durvalumab+standard of care [SoC] CRT) in patients with locally advanced, unresectable NSCLC (Stage III).

Approximately 390 patients with locally advanced, unresectable NSCLC (Stage III) will be recruited and 300 patients randomized in a 2:1 ratio to durvalumab+SoC CRT or placebo+SoC CRT. Patients will be stratified by age (<65 vs ≥65 years) and stage (IIIA vs IIIB/C).

Subjects in this study include adult subjects, ≥18 years of age with histologically or cytologically documented NSCLC who present with locally advanced, unresectable (Stage III) disease. All subjects were required to have adequate organ and marrow function.

Subjects were excluded from participation in the study if administered prior or current treatment for NSCLC, including but not limited to radiation therapy, investigational agents, chemotherapy, and mAbs.

All patients will receive one of the following platinum-based SoC chemotherapy options, in addition to radiation therapy: cisplatin/etoposide, arboplatin/paclitaxel, pemetrexed/cisplatin, or pemetrexed/carboplatin. Chemotherapy treatment regimens are outlined in Table 1.

TABLE 1
Study Treatments

| | Durvalumab | Placebo | Standard of care^{a} | |
|---|---|---|---|---|
| Study treatment | Durvalumab | Saline | Cisplatin/Etoposide | Carboplatin/Paclitaxel |
| name: | (MEDI4736) | solution | | |
| Dosage | 500-mg vial | Sterile | As sourced | As sourced locally |
| formulation:^{c} | solution for | solution of | locally | |
| | infusion after | 0.9% (w/v) | | |
| | dilution, 50 mg/mL | sodium | | |
| | | chloride for | | |
| | | injection | | |
| Route of | IV | IV | IV | IV |
| administration | | | | |
| Dosing | 1500 mg IV q4w^{e} | Saline volume | Cisplatin 50 mg/m² | Carboplatin AUC 2 and |
| instructions:^{d} | | matching | on Days 1 and 8 | Paclitaxel 40-50 mg/m² on |
| | | durvalumab | Etoposide 50 mg/m² | Day 1 weekly basis for 6 |
| | | volume | on Days 1-5 q28 | weeks - Concurrent thoracic |
| | | | days × 2 cycles + | radiotherapy |
| | | | 1 additional | Optional: paclitaxel 175-200 |
| | | | induction cycle | mg/m² and carboplatin AUC 5-6 |
| | | | optional | q 21 days given either as 1 |
| | | | Concurrent | induction cycle prior to |
| | | | thoracic | initiation of radiotherapy OR |
| | | | radiotherapy | as 1-2 consolidation cycles |
| | | | | after radiotherapy is completed |
| Packaging and | Provided in 500 mg | Sourced locally | Sourced locally | Sourced locally by site |
| labelling | vials, labelled in | by site | by site | |
| | accordance with GMP | | | |
| | Annex 13 and per | | | |
| | country regulatory - | | | |
| | requirement^{f} | | | |
| Provider | AstraZeneca | Sourced locally | Sourced locally | Sourced locally by site^{g} |
| | | by site^{g} | by site^{g} | |

| | Standard of care^{a} | | |
|---|---|---|---|
| Study treatment | Pemetrexed/ | Pemetrexed/ | Radiation |
| name: | Cisplatin ^{b} | Carboplatin ^{b} | |
| Dosage | As sourced | As sourced | As sourced |
| formulation:^{c} | locally | locally | locally |
| Route of | IV | IV | External beam |
| administration | | | radiation |
| Dosing | Pemetrexed | Pemetrexed | 5 fractions/week |
| instructions:^{d} | 500 mg/m² | 500 mg/m² and | for ~6 weeks |
| | and cisplatin | carboplatin | (±3 days) |
| | 75 mg/m² on | AUC 5 on Day 1 | (Total 60 Gy) |
| | Day 1 of each | of each 21-day | |
| | 21-day cycle, | cycle, for | |
| | for 3 cycles + | 4 cycles | |
| | 1 additional | | |
| | induction | | |
| | cycle optional | | |
| Packaging and | Sourced locally | Sourced locally | Sourced locally |
| labelling | by site | by site | by site |
| Provider | Sourced locally | Sourced locally | Sourced locally |
| | by site^{g} | by site^{g} | by site |

Patients will also receive durvalumab 1500 mg or placebo every four weeks via intravenous infusion concurrent with SoC CRT (i.e., starting on Cycle 1 Day 1 [+3 days]). Patients with complete response (CR), partial response (PR), or stable disease (SD) at 16-week tumor evaluation following completion of SoC CRT will continue to receive durvalumab/placebo as consolidation treatment (1500 mg q4w IV). Patients with RECIST 1.1-defined radiological progressive disease at the 16-week tumor evaluation following completion of SoC CRT will proceed to follow-up. Based on an average body weight of 75 kg, a fixed dose of 1500 mg of durvalumab q4w is equivalent to 20 mg/kg q4w.

The primary objective of this study is to assess the efficacy of durvalumab+SoC CRT compared with placebo+SoC CRT in terms of progression free survival per Response Evaluation Criteria in Solid Tumors version 1.1 (RECIST 1.1) as assessed by Blinded Independent Central Review (BICR). The key secondary endpoints (i.e., those included in the multiple testing procedure) are objective response rate per RECIST 1.1 as assessed by BICR, overall survival, and proportion of patients alive at 24 months from randomization (OS24).

### Example 2: Gene Expression in a Syngeneic Murine Tumor Model Demonstrating the Immunological Consequences of Radiotherapy

### Materials and Methods

Murine tumor models. Mice were housed under specific pathogen free conditions in Tecniplast 1284 IVC cages holding a maximum of 6 animals with aspenchips-2 bedding, sizzlenest nesting material, and a cardboard tunnel. Mice were housed on a 12/12 light/dark cycle and were given filtered water and fed ad libitum on Teklad Global 19% protein extruded rodent diet.

The CT26 colon adenocarcinoma cell line (purchased from ATCC in 2011) was cultured in Dulbecco's Modified Eagle's Medium supplemented with 10% (v/v) fetal bovine serum and 1% (v/v) L-Glutamine (Invivogen). Cells were not passaged for more than 3 months and were regularly screened to confirm the absence of Mycoplasma infection (PlasmoTest, Source BioScience LifeSciences, U.K.). 1×10⁵ CT26 cells were subcutaneously (s.c.) injected into the backs of Balb/c mice (Harlan Laboratories, U.K.), 1 cm from the base of the tail. Tumor volume was measured in mm³ as length×width×depth using calipers, and weight was monitored daily.

Tumor therapy. Local radiation was delivered when tumors reached 100-200 mm³. Mice were restrained in a lead shield exposing only the tumor to allow local exposure to IR with a single dose of 7 Gy using 250 kV x-rays (MXR-320/36 x-ray tube, Comet AG, Switzerland) at 12 mA and a dose rate of 2 Gy/min. Mice were sacrificed 1, 3, and 7 days post irradiation alongside time-matched non-treated controls. Tumors were harvested and used fresh for analysis by flow cytometry, with at least 20 mg of tissue from each tumor snap frozen for gene microarray analysis. For combination studies, mice received RT followed by 10 mg/kg αPD-L1 monoclonal antibody (mAb) (clone 10F.9G2, Biolegend, U.K.), dosed 3qw for 1 week and starting on day 1 of RT. Mice were sacrificed when tumors reached a volume of 1000 mm³, or for long-term surviving (LTS) mice at 100 days after therapy. Flow cytometry phenotyping studies and combination studies are representative of two independent experiments.

Exon microarray analysis. For microarray assessment, a single study was undertaken sampling 5 different tumors from each treatment group, at each time point. Fresh frozen RNA extraction was undertaken using RNAStat 60 (Amsbio, U.K.), and quality control testing of total RNA was performed using a 2100 Bioanalyzer (Agilent, U.K.). Samples were amplified using the Ovation Pico WTA System v2 (NuGEN Technologies, Netherlands). Following QC testing, cDNA was fragmented and labelled using the Encore Biotin Module (NuGEN Technologies, Netherlands), which was then hybridized to mouse exon arrays according to NuGEN guidelines for Affymetrix GeneChip® arrays. Microarray analysis was performed using the Mouse Exon 1.0 ST array (Affymetrix, U.K.). All microarray data have been deposited into GEO (accession number GSE74875).

Data analysis. Raw microarray data underwent Robust Multichip Algorithm (RMA) pre-processed/normalized on core transcript probes (Bolstad et al., Bioinformatics 19(2): 185-93 (2003)). Quality control was then performed excluding three outliers after data integrity assessment (one from each treatment group for day 1 and one from the radiation treated group at day 3). Affy AFFX control transcripts were removed along with uninformative transcripts (log 2 expression threshold <3.6473 and variance threshold <0.0088). 8500 reliably detected transcript Ids (Affymetrix transcript cluster Ids) remained for analysis.

Comparisons were performed on the treatment groups (untreated versus irradiated) per time point and differentially expressed transcript Ids (up or downregulated) were identified using a cut-off p-value of 0.05 (ANOVA). The median log 2 intensity value for each transcript Id per time point was calculated for the untreated tumor groups and then subtracted from treated samples at their equivalent time point giving control normalized transcript expression intensities per time point. Hierarchical Cluster Analysis (HCA); unnormalized, linkage=Ward and distance=uncentered correlation (Omicsoft ArrayStudio) was performed to cluster the control normalized transcript expression data per sample and transcript Id. Pathway categorized differentially regulated gene-set data were plotted per time point as bubble diagrams (MatLab) with the color of the bubble indicating the direction of gene regulation. The size of the bubble indicates the absolute fold change expression values of each pathway. Mouse gene annotations were assigned to transcript Ids using BioMart (Mus Musculus genes GRCm38.p2) and IDconverter (Alibes et al., BMC Bioinformatics 8:9. doi: 10.1186/1471-2105-8-9 (2007)). Functional enrichment and network analyses were performed using Ingenuity Pathway Analysis (IPA, Ingenuity® Systems). Transcripts that were upregulated or downregulated by at least 1.5-fold were mapped to pathways and to upstream regulators. Gene Ontology enrichment analysis was performed with the g:GOSt function in g: Profiler (Reimand et al., (2011 update), Nucleic Acids Res. 39(Web Server issue): W307-15. doi: 10.1093/nar/gkr378 (2011)).

Flow cytometry. Tumors were cut into 1 mm³ pieces and incubated at 37° C. for 40 minutes in 2 U/mL DNAse (Sigma, U.K.), 300 CDU/mL collagenase I (Life Technologies, U.K.) and 0.9 mg/mL dispase II (Sigma, U.K.) in PBS, and pushed through a 100 μm cell strainer with FACS buffer (PBS with 10% FCS). Expression of CD4, CD8 (BD Biosciences, U.K.), CD11b, CD11c, CD45, CD69, CD86, CD206 (Biolegend, U.K.), MHC-II, F4/80, Gr1, NKp46, B220, PD-1, and CTLA-4 (all from eBiosciences, U.K. unless otherwise stated) were analyzed by flow cytometry following incubation with CD16/CD32 Fc blocking antibodies (Life Technologies, U.K.). A viability stain (Life Technologies, U.K.) was included to exclude dead cells. Regulatory T-cells were analyzed using the Mouse Regulatory T-cell Staining Kit #3 (eBioscience, U.K.)

Statistical analysis. Mann Whitney U tests were used to compare flow cytometry data and tumor volume between two groups. Gene expression profiling data were assessed as described above. Log-Rank Mantel-Cox tests were performed on survival data. Data were considered significantly different if P<0.05.

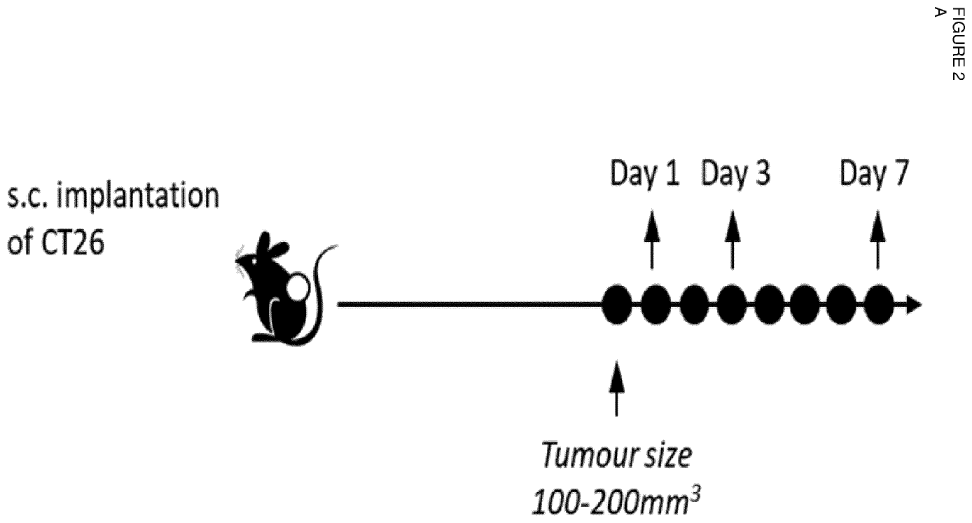

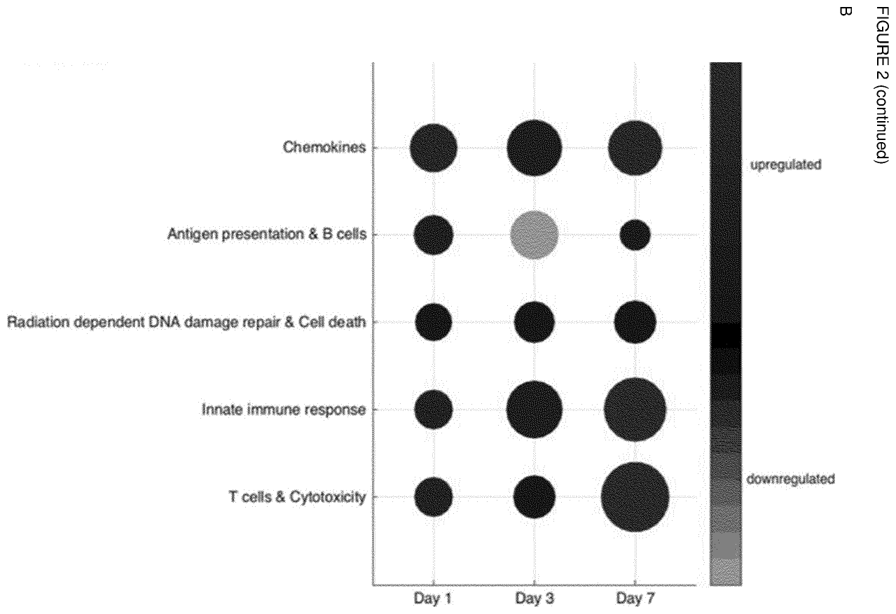

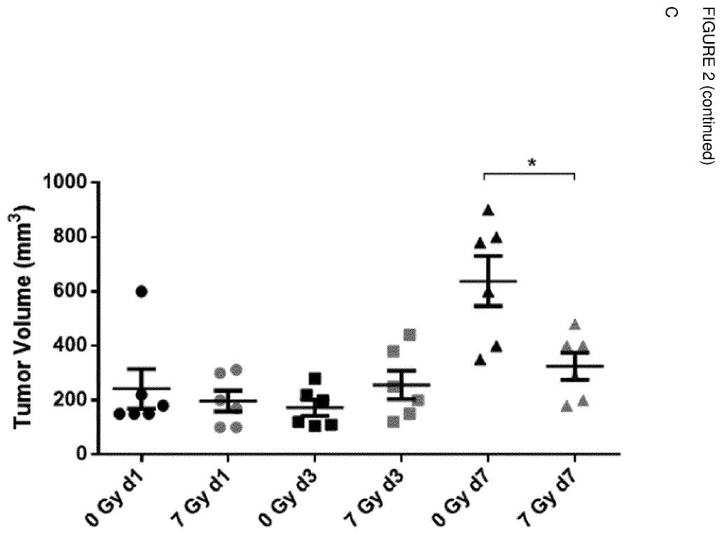

RT leads to activation of innate and adaptive immunity. Immunocompetent Balb/c mice bearing established CT26 tumors received a single 7 Gy dose of RT and tumors were excised 1, 3, and 7 days post-treatment (FIGS. 2A and 2C) for exon microarray analysis to identify early transcriptional changes. The transcriptomes of irradiated tumors were compared to those of non-treated (NT) time-matched controls resulting in the identification of 757 genes (+/−1.5 fold change and p<=0.05) that were significantly differentially expressed (up or downregulated) for at least one of the time points (Table 2). In addition to the expected p53 activation pathway (radiation-dependent DNA damage and cell death), IPA functional enrichment analysis of the data highlighted a strong bias for innate and adaptive immune functions. These included antigen presentation, T-cell activation and cytotoxicity, and chemokine production (FIGS. 2B, and 3, and Tables 3 and 4). It was noteworthy that the number of differentially regulated genes clearly increased from day 1 to day 7 (Table 2).

TABLE 2
Number of up- or down-regulated genes meeting
the cut-off threshold (≥+/−1.5-fold
change and p ≤ 0.05, ANOVA) at the different time points.

| | Day 1 | Day 3 | Day 7 |
|---|---|---|---|
| ≥1.5 fold change | 205 | 275 | 503 |
| ≤−1.5 fold change | 185 | 242 | 205 |
| Total differentially | 390 | 517 | 708 |
| regulated genes per | | | |
| day | | | |

TABLE 3

| | | | −log(p |
|---|---|---|---|
| | Pathway | Mouse Genes | value) |
| Day 1 | Antigen presentation pathway | Psmb9, Ifng, H2-Q4, H2-Aa, Psmb8, Cd74, Tap1, H2-DMa, H2- | 18.6 |
| | | DMb1, H2-DMb2, Tapbp, H2-Eb1 | |
| | Communication between innate | Ifng, Cd40lg, H2-Q4, II15, Cd8a, Cd8b1, Cxcl10, Ccl4, Ccl3, II1b, | 14.4 |
| | and adaptive immune cells * | Tnf, H2-Eb1 | |
| | Dendritic cell maturation * | Cd40lg, H2-Q4, II15, H2-Aa, H2-Ab1, H2-DMa, H2-DMb1, H2- | 9.36 |
| | | DMb2, II1b, Stat2, Stat1, Tnf, H2-Eb1, Fcgr4 | |
| | T helper cell differentiation | Ifng, Cd40lg, H2-DMa, H2-DMb1, H2-DMb2, H2-Aa, H2-Ab1, | 9.11 |
| | | Stat1, Tnf, H2-Eb1 | |
| Day 3 | Granulocyte adhesion and | II1a, VCAM1, Cxcl11, Ppbp, II1rl1, Cxcl12, Ccl17, Mmp13, Ccl5, | 11.9 |
| | diapedesis * | Cldn6, Cxcl10, Cxcl16, Cxcl3, Ccl4, II1rn, Ccl3, Ccl2, Cxcl14, | |
| | | Cxcl1, Ccl7 | |
| | Agranulocyte adhesion and | II1a, Vcam1, Cxcl11, Ppbp, Cxcl12, Ccl17, Mmp13, Ccl5, Cldn6, | 10.5 |
| | diapedesis * | Cxcl10, Cxcl16, Cxcl3, Ccl4, II1rn, Ccl3, Ccl2, Cxcl14, Cxcl1, | |
| | | Ccl7 | |
| | Communication between innate | Cxcl10, II1a, Ccl4, Cd80, II1rn, Ccl3, II15, Ccl5, Tlr3, H2-Eb1 | 8.5 |
| | and adaptive immune cells * | | |
| | Dendritic cell maturation * | II1a, II15, H2-Aa, Col10a1, H2-Ab1, Cd80, H2-DMa, II1rn, | 7.63 |
| | | Pik3cg, H2-DMb1, H2-DMb2, Stat2, Tlr3, Stat1, H2-Eb1, Col3a1 | |
| Day 7 | Granulocyte adhesion and | Sell, Sele, Cxcl11, Cxcl12, Thy1, Mmp2, Ccl5, Ccl11, Cldn6, | 11.9 |
| | diapedesis * | Itgal, Ccl9, Selplg, II1r2, Cxcl10, Ccl4, Selp, II1rn, Ccl3, Ccl2, | |
| | | Tnf, Ccl6, Ccl7, Mmp19, Itga4 | |
| | Communication between innate | Ifng, II15, Ccl5, Cd8a, Tlr9, Cd8b1, Ccl9, Cxcl10, Cd28, Ccl4, | 8.98 |
| | and adaptive immune cells * | II1rn, Ccl3, Tlr3, Tnf | |
| | Agranulocyte adhesion and | Sell, Sele, Cxcl11, Cxcl12, Mmp2, Ccl5, Ccl11, Cldn6, Ccl9, | 8.92 |
| | diapedesis * | Selplg, Cxcl10, Ccl4, Selp, II1rn, Ccl3, Ccl2, Tnf, Ccl6, Ccl7, | |
| | | Mmp19, Itga4 | |
| | T Cell receptor signaling | Camk4, Prkcq, Cd3e, Cd8a, Cd8b1, Cd3d, Ctla4, Ptprc, Cd28, | 8.54 |
| | | Cd3g, Lck, Txk, Card11, Grap2, Lat, Itk | |
| | Role of pattern recognition | Ifng, Oas1a, Oas1g, Prkcq, C3, Oas2, Ccl5, Oas3, Tlr9, Ifih1, | 7.24 |
| | receptors in recognition of | Irf7, Ddx58, Casp1, Prkch, Eif2ak2, Tlr3, Tnf | |
| | bacteria and viruses | | |
| | Crosstalk between dendritic cells | Ifng, Cd28, Prf1, Klrd1, II15, Cd226, Ltb, Tnfsf10, Tlr3, Tlr9, Tnf, | 6.68 |
| | and natural killer cells | Itgal | |
| | Interferon signaling | Ifit3, Ifng, Oas1a, Oas1g, Ifi35, Stat2, Irf9, Psmb8, Stat1 | 6.33 |

Key predominant immunological pathways (as analyzed by IPA software) associated with differentially expressed genes (fold change = +/−1.5 and p ≤ 0.05) at the three time points (day 1, 3, and 7) in CT26 tumors following 7 Gy IR.
* pathways implicated at multiple time points. Non-disease related immune pathways present in the top 10 with the most significant p values are highlighted.

TABLE 4

| | | | Day 1 (fold | Day 3 (fold | Day 7 (fold |
|---|---|---|---|---|---|
| | Gene name | Cluster | change) | change) | change) |
| T cells & | Cd3d | A | 1.84 | −2.47** | 3.97*** |
| Cytotoxicity | Cd3g | A | 1.37 | −1.94 | 3.35*** |
| | Cd3e | A | 1.46 | −1.72 | 3.09*** |
| | Cd8a | A | 2.16* | −1.69 | 3.38*** |
| | Cd28 | A | −1.05 | −1.24 | 3.39*** |
| | Pdcd1 (Pd1) | A | −1.05 | −1.25 | 1.95*** |
| | Ctla4 | A | 1.07 | −1.09 | 2.54*** |
| | Icos | A | 1.03 | −1.17 | 1.78*** |
| | Lag3 | A | 1.01 | 1.04 | 2.24*** |
| | Entpd1 (Cd39) | A | 1.38 | −1.09 | 1.63*** |
| | Havcr2 (Tim3) | A | −1.05 | 1.04 | 2.54*** |
| | Ifng | A | 1.67** | 1.24 | 1.99*** |
| | Il15 | B | 1.61*** | 2.65*** | 2.14*** |
| | Tnfsf9 (Cd137) | B | 1.15 | 1.5* | 1.12 |
| | Cd274 (Pd-11) | C | 2.41 | 2.29*** | 2.11*** |
| | Tnf | C | 1.53* | 1.1 | 1.71** |
| | Sell (Cd621) | C | 1.48 | 1.39 | −1.6** |
| | Cd40lg | C | 1.7** | −1.17 | −1.47 |
| | Gzmb | C | 2.58* | 3.75*** | 4.04*** |
| Innate | Tlr9 | A | 1.24 | 1.16 | 1.68*** |
| immune | C2 | A | 1.32 | 1.1 | 1.84*** |
| response | Stat4 | A | 1.05 | −1.36 | 2.25*** |
| | Tlr3 | B | 1.21 | 2.21*** | 2.24*** |
| | Nos2 | B | 1.46 | 1.82*** | 1.79*** |
| | Cfb | B | 1.35 | 1.44 | 2.66*** |
| | Irf7 | B | 1.27 | 2.77*** | 4.39*** |
| | Irf9 | B | 1.22 | 1.39 | 1.71*** |
| | Mx1 | B | 1.53 | 3.68*** | 5.41*** |
| | Oas1a/Oas1g | B | 1.42 | 1.92*** | 2.82*** |
| | Oas2 | B | 1.27 | 2.15** | 2.54*** |
| | Irf1 | C | 2.14*** | 1.41 | 1.49 |
| | Stat1 | C | 2.08*** | 1.61* | 1.85*** |
| | Stat2 | C | 1.64* | 2.05*** | 2.38*** |
| | C3 | C | 1.98*** | 1.42 | 2.24*** |
| | C1ra | C | 1.37 | 1.48 | 1.87*** |
| | C1rb | C | 1.37 | 1.48 | 1.87*** |
| | Cd74 | D | 2.21** | −2.02** | −1.19 |
| | Retnla | D | −1.55 | −6.93*** | 2.54 |
| | Mrc1 | E | −1.34 | −1.89*** | −1.41 |
| | Cd163 | E | −1.58** | −1.96*** | 1.09 |
| Radiation | Tnfsf10 (Trail) | A | 1.22 | 1.62* | 1.63*** |
| dependent | Casp4 | B | 1.54*** | 2.09*** | 1.85*** |
| DNA | Parp11 | B | 1.24 | 1.79*** | 2.01*** |
| damage | Cdkn1a | C | 1.46 | 1.7*** | 1.24 |
| repair & Cell | Fas | C | 1.8*** | 1.52* | 1.31 |
| death | Parp14 | C | 1.53 | 1.8* | 2.51*** |
| | Parp12 | C | 1.32 | 1.76** | 2.06*** |
| | Parp9 | C | 1.44 | 1.72*** | 1.97*** |
| | Brca1 | E | −1.07 | −1.2 | −1.59*** |
| | Brca2 | E | −1.04 | −1.18 | −1.59*** |
| | Ercc1 | E | −1.02 | −1.13 | −1.77*** |
| | Lig4 | E | −1.83* | −1.59 | −1.33 |
| | Pola1 | E | −1.09 | −1.32 | −1.56*** |
| | Gadd45a | E | −2.13*** | −1.53* | −1.2 |
| | Parpbp | E | 1.33 | −1.11 | −1.56* |
| | Hist1h4h | E | −1.87*** | −1.81*** | −1.35 |
| | Hist2h4 | E | −1.72* | −1.83** | −1.06 |
| Antigen | cd80 | B | 1.26 | 1.87*** | 1.37 |
| presentation | Psmb8 | C | 1.84*** | 1.4 | 1.52* |
| & B cells | Psmb9 | C | 2.03*** | 1.43 | 1.4 |
| | Psmb10 | C | 1.59* | 1.55* | 1.51* |
| | Tap1 | C | 1.85*** | 1.37 | 1.48 |
| | Tapbp | C | 1.51** | 1.6*** | 1.6*** |
| | H2-DMa | D | 1.6** | −1.54** | −1.05 |
| | H2-DMb1 | D | 1.61* | −1.9*** | −1.24 |
| | H2-DMb2 | D | 1.61* | −1.9*** | −1.24 |
| | H2-Aa | D | 1.95* | −2.28*** | 1.02 |
| | H2-Ab1 | D | 1.66* | −1.72* | 1.01 |
| | H2-Eb1 | D | 2.08* | −2.43*** | −1.02 |
| | Prkcb | D | −1.26 | −2.48*** | 1.32 |
| | Cd24a | D | 1.02 | −2.32* | 1.31 |
| | Cd93 | D | −1.03 | −1.73*** | −1.13 |
| | Cd38 | D | 1.59** | −1.09 | 1.26 |
| | Cd209d | D | −1.18 | −2.02*** | 1.12 |
| | Cd209c | D | −1.08 | −1.96*** | −1.04 |
| Chemokines | Cxcl10 | B | 2.12* | 3.04*** | 2.58*** |
| | Ccl2 | B | 1.38 | 1.9*** | 1.51** |
| | Ccl5 | B | 1.44 | 1.75* | 4.17* |
| | Ccl6 | B | 1.08 | 1.33 | 1.51* |
| | Ccl7 | B | 1.25 | 1.63** | 1.97*** |
| | Cxcl1 | C | 2.58*** | 2.41*** | 1.26 |
| | Cxcl2 | C | 1.59 | 3.63*** | 1.15 |
| | Cxcl11 | C | 2.7** | 3.1*** | 2.72*** |
| | Ccl3 | C | 2.4** | 2.28** | 2.63*** |
| | Ccl4 | C | 2.37*** | 2.48*** | 3.45*** |
| | Ccl11 | D | 1.04 | −1.29 | 3.24*** |
| | Ccr2 | D | 1.22 | −1.56* | 1.3 |
| | Cxcl9 | D | 4.25*** | −1.02 | 1 |
| | Cxcl12 | D | 1.38 | −1.64* | 1.77* |
| | Cxcl14 | D | −1.07 | −2.26*** | 1.38 |
| | Cxcl16 | D | 1.37 | −1.57*** | −1.11 |
| | Ccl17 | F | −1.21 | −1.56** | −1.18 |

Classification of key genes in each cluster (A-E) from the heatmap (FIG. 2) into functional sets with immune or radio-modulatory associations. Gene expression fold change at each time point (radiation treated versus untreated tumor) are shown, significant p values are shown with asterisks (p ≤ 0.05*; p ≤ 0.01**; p ≤ 0.005*** ). Functional classifications were guided by the Nanostring nCounter Mouse PanCancer Immune Profiling Panel and author knowledge.

The pattern of response is also evident in the network maps built out from the top immune related upstream regulators that are significantly differentially regulated at each time point (FIG. 3). Furthermore, pathway analysis highlighted that 30.8% of the up and downregulated genes were associated with immune system processes (GO:0002376) giving the most significant enrichment p value of 4.35E-72 seen with this gene-set emphasizing the predominance of immune processes in the 7 days after irradiation. However, just over 5% of genes showed similar behavior across all 3 time points (38 up/3 down), thus highlighting different phases in the radiation response at the 3 time points assessed.

A detailed hierarchical cluster analysis (HCA) of the differentially expressed genes identified 5 clusters each containing genes co-regulated at the different time points (FIG. 2B). Genes in cluster A were significantly upregulated at day 7 when compared to expression in control tumors. Pathway analysis demonstrated that the vast majority of these genes are associated with adaptive immune cell responses, including T-cell receptor signaling and CD28 signaling. Illustrating this point is the increase in Cd3d/e/g, Cd8a and Cd28 expression, suggesting an increased infiltration or expansion of CD3⁺/CD8⁺ T-cells. Signs of an active anti-tumor immune response were also suggested by the upregulation of Ifnγ. Another upregulated gene of interest is Tnfsf10, which encodes the cell death receptor TRAIL. Radiation is known to induce the tumor cell surface expression of cell death receptors and their ligands, and upregulation of TRAIL on T-cells can help direct tumor cell killing via activation of the TRAIL receptor. Interestingly, the observed concurrent expression of inhibitory immune checkpoints, such as Pd-1, Lag3, and Ctla4, suggested that this adaptive immune response could be transient. The development of this immunosuppressive tumor microenvironment is also reinforced by the up-regulation of Cd39/Entpd1, which in association with the enzymatic activity of Cd73 is known to contribute to adenosine-dependent immune cell suppression.

The second cluster (B) was not only enriched with genes associated with innate immunity but also with genes encoding proteins involved in the communication between the innate and adaptive arms of the immune system. These genes were continuously upregulated during the course of the experiment. Some genes were significantly upregulated at day 1, but the expression of the majority of the genes was significantly upregulated from day 3. This expression pattern suggests that the innate immune response is initiated early and remains relatively constant during the first week following a single dose of RT. Relevant genes in this cluster included (d80, which is a co-stimulatory receptor expressed on APC, and IL15, a cytokine expressed by monocytes and dendritic cells that acts as a potent inducer/activator of natural killer cells and T-cells. In addition, cluster B contained Nos2 (an enzyme induced by IFNγ on activated macrophages), Cfb (complement factor B of which the catalytic subunit Bb can activate C3 convertase to subsequently activate B Cells) and the toll like receptor 3 (Tlr3). Furthermore, type I and type II interferon-regulated genes including Irf7, Irf9, Mx1, Oas1a/g and Oas2, and chemokine genes Cxcl10, Ccl2/5/6 and 7 were enriched in this cluster.

Cluster C's profile is more consistent across timepoints and could be considered as the “first gene cluster” in terms of chronological response. It comprised a large set of genes significantly upregulated at the earlier timepoints (day 1 and 3) with a smaller subset being upregulated later (day 3 to day 7). Innate immune response, radiation dependent DNA damage repair and cell death, and chemokines were the predominant functional enrichments in this cluster but several genes associated with T-cells and cytotoxicity, and antigen presentation and B-cells were also present. Genes associated with innate immunity included those encoding complement, such as C3, C1ra, and C1rb, and interferon regulated genes including Stat1. Interestingly, Stat1 was highlighted as an upstream regulator in the IPA functional enrichment analyses at all three time points (FIG. 3, and Table 4). Chemokines such as Ccl4 (a chemokine associated with the recruitment of macrophages and NK cells) were also strongly upregulated at day 1; as were genes associated with antigen processing (Psmb8/9/10, Tap1 and Tapbp). Radiation dependent DNA damage repair and cell death and T-cell & cytotoxicity were the predominant functional enrichments in this cluster. Noteworthy and validating the dataset, is the expression of genes associated with p53 signaling, which is linked to the direct effects of IR. Cellular stress such as DNA damage caused by IR activates p53, inhibits cell proliferation and primes tumor cells for apoptosis. For example, p53 activation induces the expression of the inhibitor of cell cycle progression gene Cdkn1a (1.7 fold upregulated at day 3, p<0.01). Similarly, the death receptor Fas (1.8 fold upregulated at day 1, p<0.01) is known to be induced by activated p53 and renders cells sensitive to FAS-ligand expressing immune effector cells. Relevant upregulated genes associated with adaptive immunity included Cd40lg (usually expressed on activated CD4⁺ T-cells), the immune checkpoint protein Pd-l1 (Cd274), which is significantly upregulated from day 3, and Gzmb (Granzyme B) which, in addition to its function in target cell killing, is involved in basement membrane remodeling and lymphocyte transmigration, and was significantly upregulated from day 1 onwards.

Genes in Cluster D were transiently upregulated at day 1 before being significantly downregulated at day 3. Pathway analysis demonstrated enrichment of genes associated with antigen presentation (H2 genes, Cd74, Cd209d, and Cd209c) and B-cell activation (Cd24a), along with chemokine expression (Cxcl9 and Cxcl12). None of the antigen presentation and B-cell associated genes were significantly differentially regulated by day 7, suggesting that the cascade of effects had progressed further down the immune functional pathways.

The final cluster identified (Cluster E) was the largest cluster, containing 180 genes. Radiation dependent DNA damage repair is the key functional enrichment with significant downregulations apparent for certain genes as early as day 1 (day 1 Lig4; day 1 and 3 Gadd45a or day 7 Brca1, Brca2, Ercc1, Pola1, and Parbp). This data supports previous studies that reveal rapid repair of DNA within 24 hours post-irradiation. Finally, a strong link between cholesterol biosynthesis downregulation and radiation was identified.

Altogether, the transcriptomic analysis confirms that a single dose of radiation to CT26 tumors triggers p53-dependent cell death. Triggering cell death is probably a rate-limiting step to initiate both innate and adaptive immune responses in irradiated tumors. Indeed, the gene expression profile suggests that RT-induced tumor cell death leads to the recruitment and activation of innate immunity (IFNα expression, antigen processing/presentation, macrophage recruitment and dendritic cell maturation) followed by activation of the adaptive immune response (IFNγ signaling, T-cell cytotoxicity, T-cell receptor signaling, and B-cell activation). However, as for any physiological system, this biological immune response is expected to be transient, as demonstrated by increased expression of several immunosuppressive molecules.

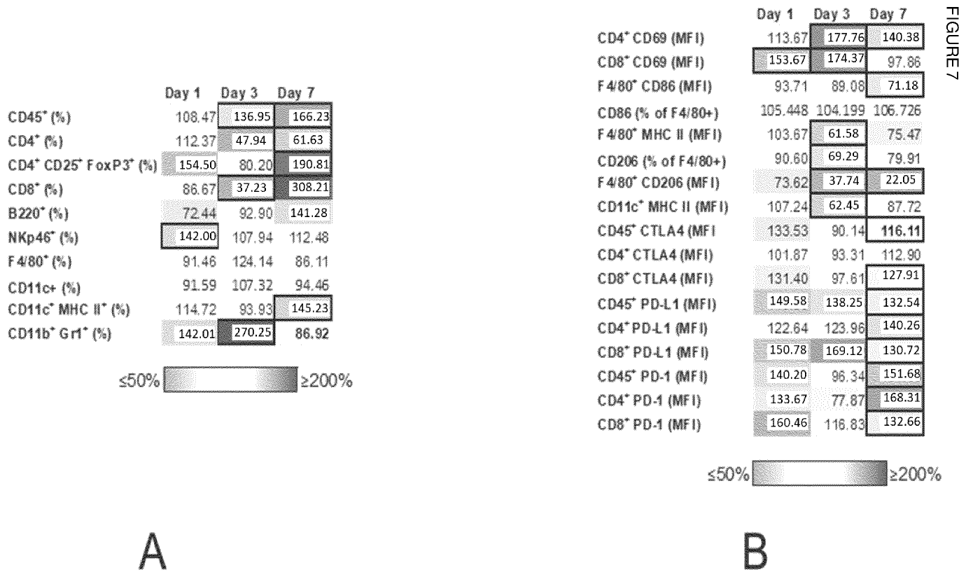

To validate the gene microarray data and to further explore phenotypic changes in immune components following IR at the protein level, the same tissues were analyzed in parallel by flow cytometry. A heat map showing the fold changes in lineage and phenotype markers from the matched tumor tissue is summarized in FIG. 7.

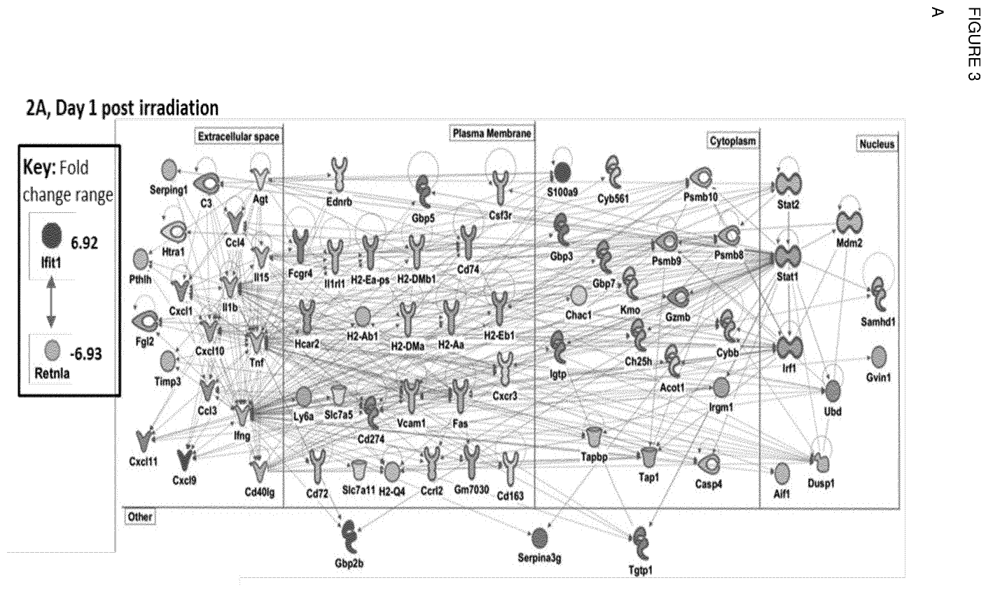

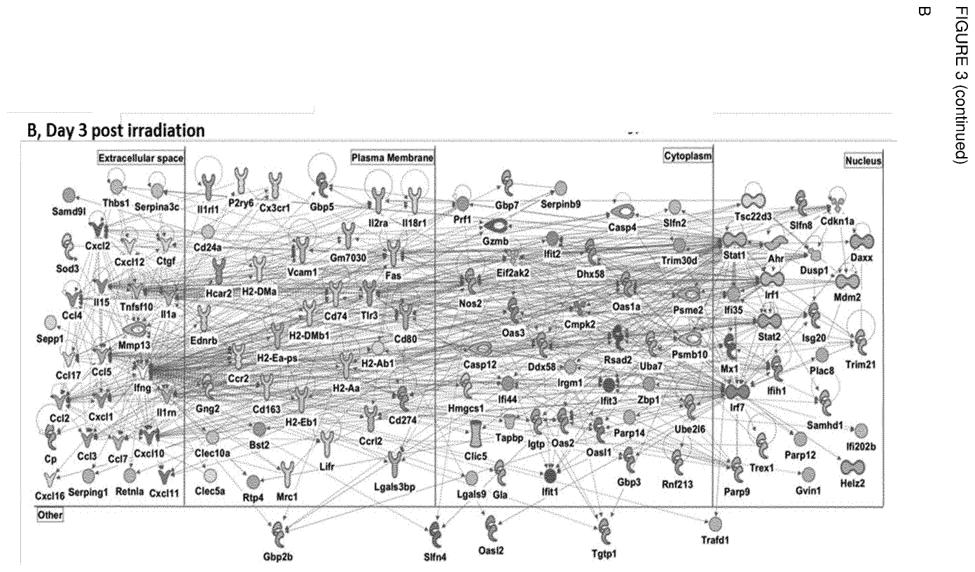

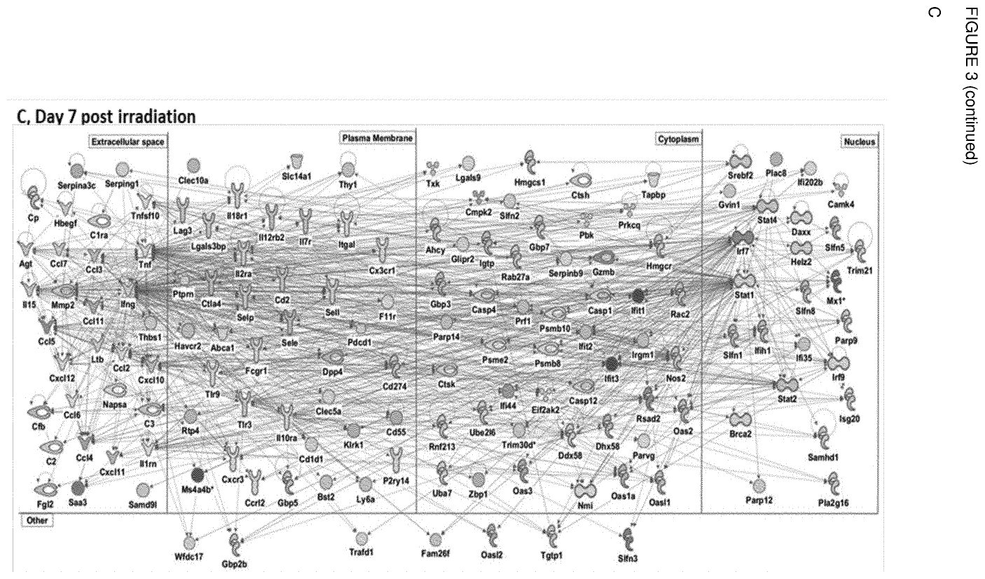

RT modifies the phenotype of tumor-infiltrating myeloid cell populations. Macrophages show a high degree of lineage plasticity, however tumor-associated macrophages (TAMs) are predominantly skewed towards an M2 phenotype in a number of cancer types. M2 cells express CD206 (also known as mannose receptor or MRC1), are poor APCs, and may contribute to immune escape and disease progression through the release of pro-angiogenic and immunosuppressive factors. In contrast, M1-differentiated macrophages co-express co-stimulatory molecules such as CD86, enabling efficient lymphocyte activation. Both the frequency and differentiation state of TAMs in RT-treated and time-matched NT control tumors were analyzed. Whilst the overall number of F4/80⁺ TAMs did not alter significantly following RT (FIG. 4A), the expression of CD86 and CD206 were modulated. Seven days after RT, F4/80⁺ cells had reduced expression of CD86, relative to NT time-matched controls (MFI of 695.0±46.6 NT vs. 494.7±13.9 irradiated, P<0.05; FIG. 4B). Moreover, CD206 expression was significantly reduced on tumor-infiltrating F4/80⁺ cells 3 days after RT (MFI of 263.7±23.8 in NT vs. 99.5±8.1 in irradiated tumors. P<0.001; FIG. 4C). This reduced expression was also observed at day 7 post RT. A similar pattern of response was also observed in the transcriptomic analysis (Table 4). The ratio of CD86⁺ to CD206⁺ positive F4/80⁺ cells increased at day 3 relative to time-matched NT controls (1.8±0.05 NT vs. 2.7±0.12 irradiated, P<0.01) (FIG. 4D). In addition to changes in the expression of CD86 and CD206, a shift in macrophage phenotype is also suggested by the increase in Nos2 and Stat1 expression observed in the gene profiling data following irradiation (FIG. 3 and Table 4). Furthermore, a decrease in gene expression of resistin-like alpha (Retnla) and Cd163 (6.9- and 2-fold, respectively), which are linked to a M2-like phenotype, was also identified 3 days post RT (FIG. 3 and Table 4). Given that no change in overall TAM number was found these data suggest that RT leads to a transient polarization of macrophages towards an M1 phenotype.

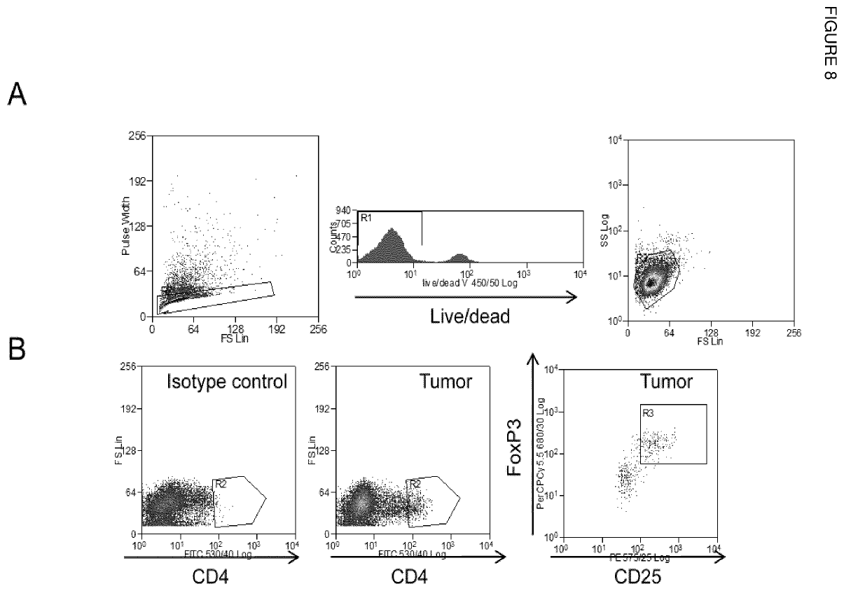

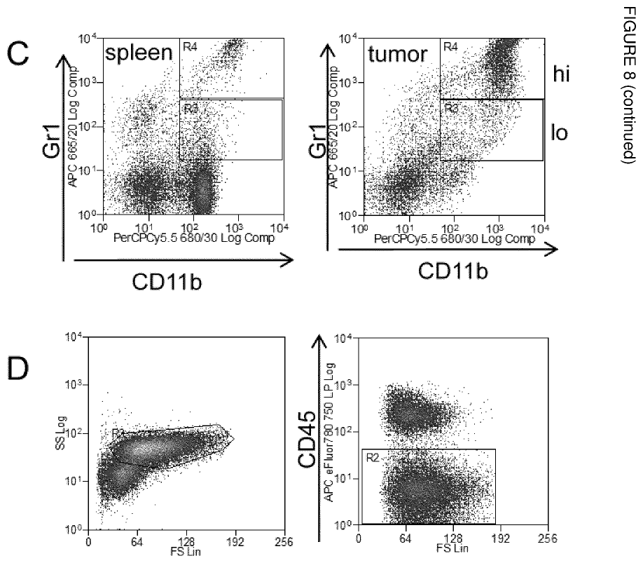

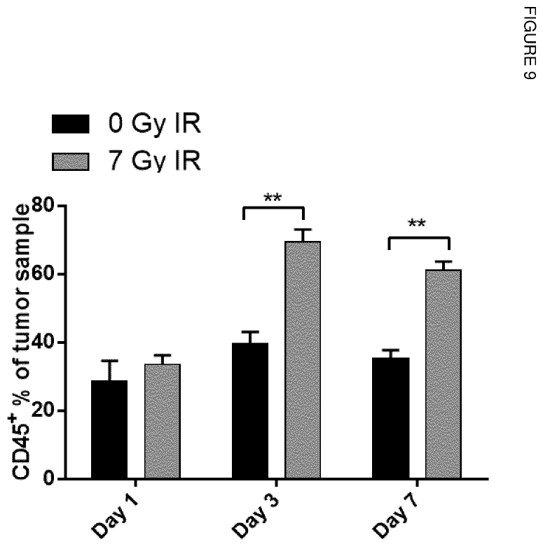

Myeloid-derived suppressor cells (MDSCs) have the capacity to suppress anti-tumor immune responses and therefore may impact the immunogenicity of RT. Whilst no changes in the frequency of tumor-infiltrating CD11b⁺Gr1^{lo} cells were observed at any of the time points following RT (FIG. 4E and gating strategy FIG. 8), a 2.7 fold increase in CD11b⁺Gr1^{hi} cells (phenotypically defined as MDSCs) was observed at day 3 in RT-treated tumors (FIG. 4F). This increase appeared transient as by day 7 no significant difference in the frequency of CD11b⁺Gr1^{hi} cells was observed between the NT and RT-treated groups.

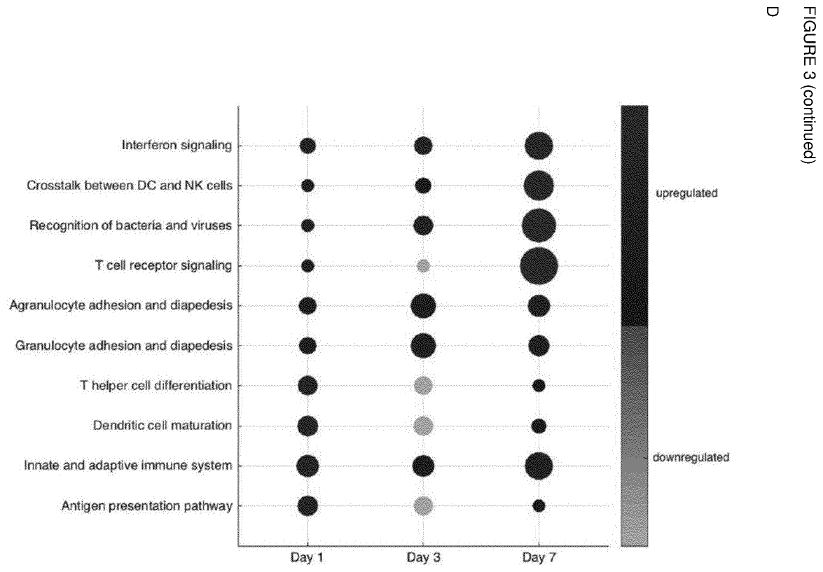

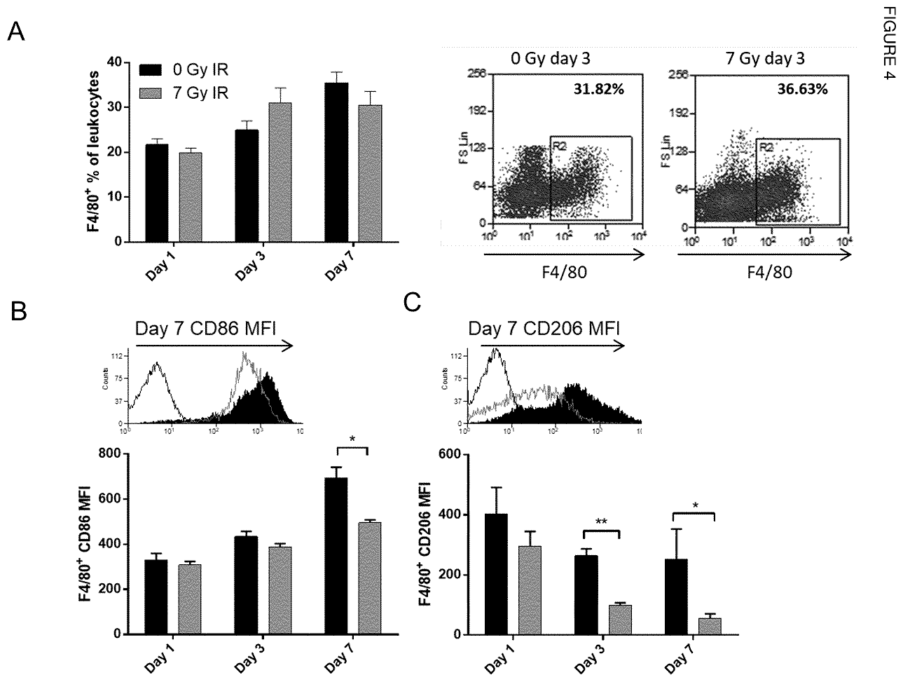

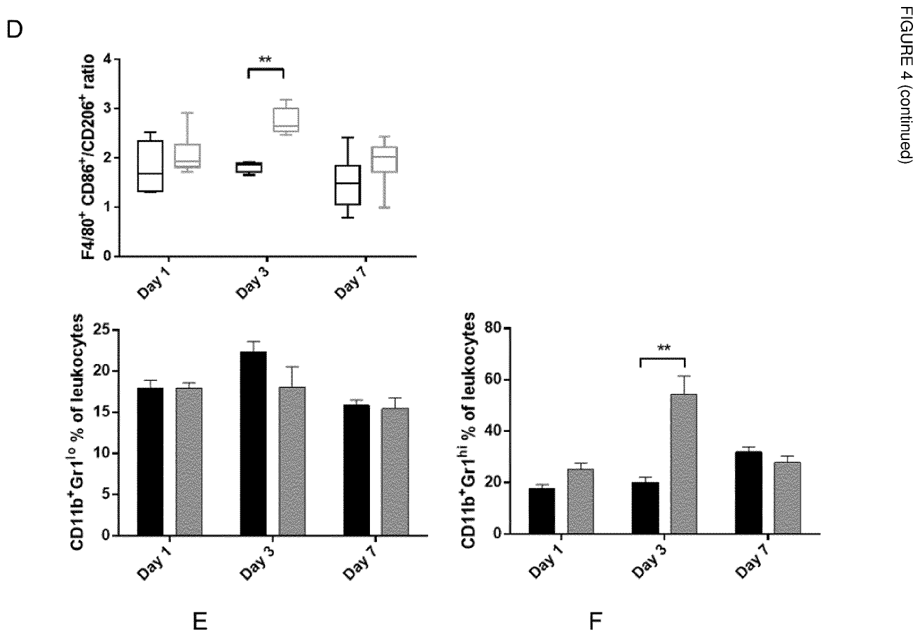

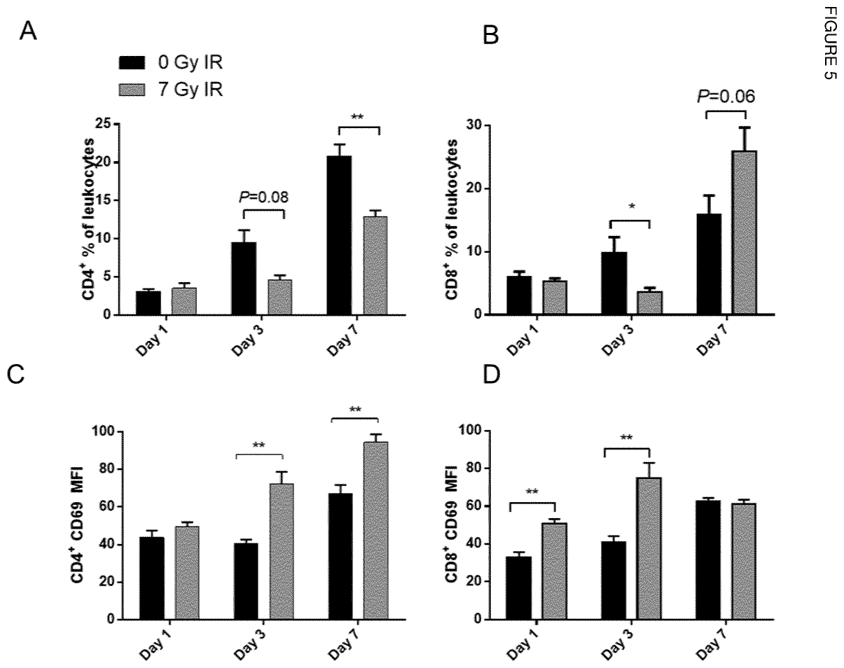

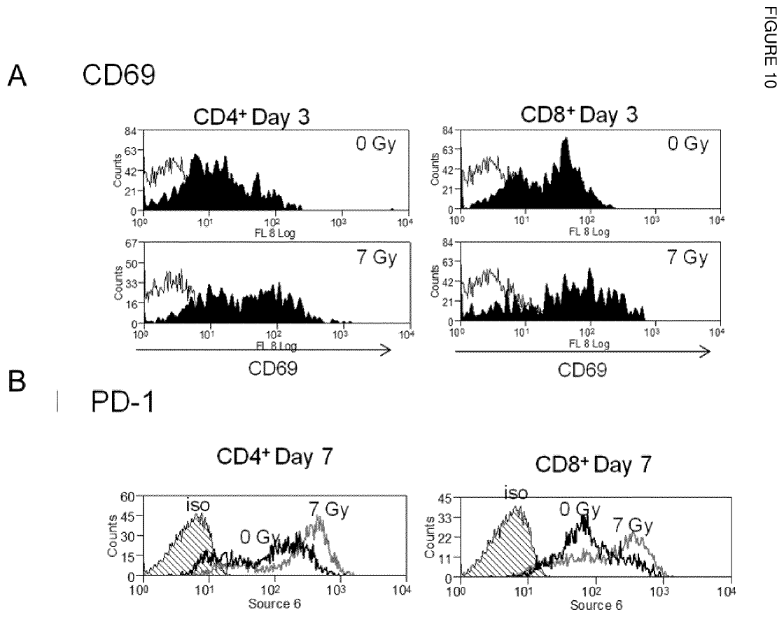

RT leads to T-cell activation and alters the CD8:Treg ratio in tumors. Although RT led to an overall increase in the proportion of CD45⁺ cells infiltrating the tumor (FIG. 9) CD4⁺ and CD8⁺ T-cell numbers were found to be reduced by 52% and 63% respectively 3 days after RT when compared to time-matched controls (FIGS. 5A and 5B). Whilst CD4⁺ T-cell numbers remained depleted 7 days after treatment, CD8⁺ T-cell numbers had recovered and there was a strong trend towards expansion in RT-treated tumors (15.9±3.0% to 25.9±3.8%. P=0.06). Interestingly, elevated expression of the early activation marker CD69 was observed on the remaining tumor-infiltrating CD4⁺ (at day 3 and 7 post RT) and CD8⁺ (at day 1 and 3 post RT) T-cells relative to time-matched controls (FIGS. 5C and 5D and FIG. 10A) suggesting that RT had induced T-cell activation.

Even in NT tumors the proportion of CD4⁺ cells with a Treg phenotype increased 3.3 fold from day 1 to day 7 (FIG. 5E) demonstrating how the tumor microenvironment changes over time. However, comparison of time-matched tumors revealed that RT further increased the frequency of Tregs by an additional 32% when assessed at 7 days post treatment. Despite this, the CD8:Treg cell ratio, often reported to be associated with better prognoses in cancer, was 2.5 fold higher in RT-treated tumors when compared to the day 7 NT controls (FIG. 5F).

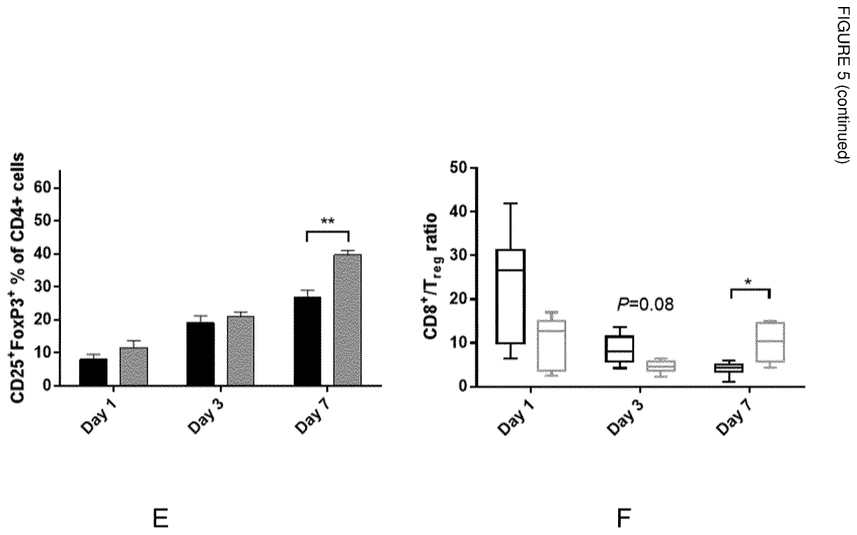

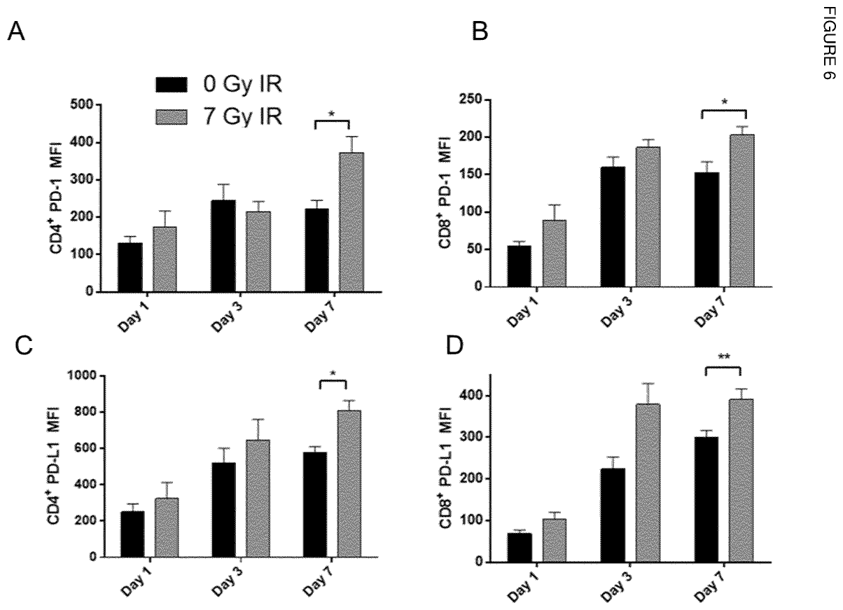

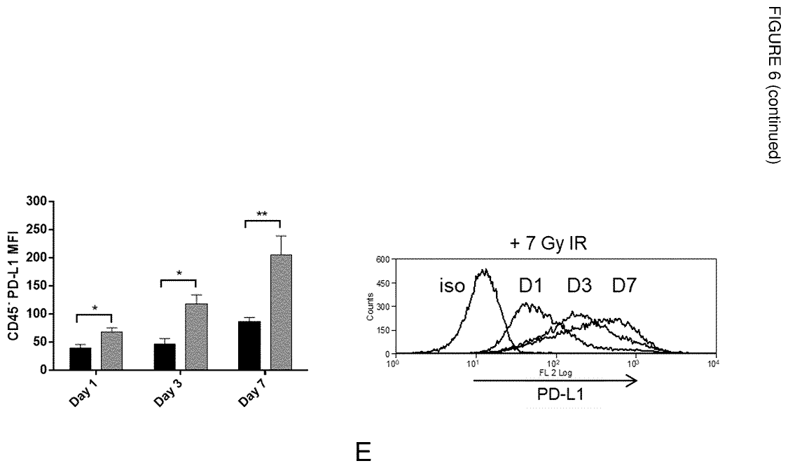

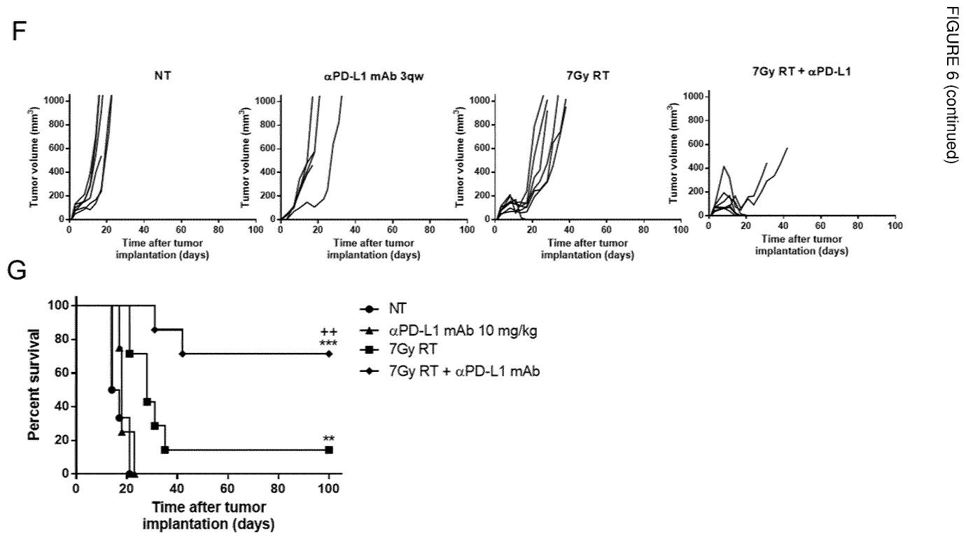

RT leads to expression of PD-1 and PD-L1 in the tumor microenvironment which limits anti-tumor efficacy. The gene-profiling data revealed that RT led to increased expression of several co-inhibitory immune checkpoints in the tumor. Given the consistent upregulation of Pd-l1 observed following RT at the mRNA level and expression of Pdcd1 (PD-1) at day 7 (FIG. 3 and Table 4), initial studies focused on the PD-1/PD-L1 pathway. Flow cytometry permitted further contextualisation of the mRNA data and revealed increased expression of both PD-1 and PD-L1 on CD4⁺ and CD8⁺ T-cells 7 days after RT (FIGS. 6A-6D and FIG. 10B). Moreover, RT also led to increased expression of PD-L1 on tumor cells at all time points tested (FIG. 6E). This data closely resembled the expression patterns observed at the mRNA level (FIG. 3 and Table 4).

Therapeutic studies were undertaken to determine whether blockade of the PD-1/PD-L1 axis would impact the anti-tumor efficacy of RT. Mice received RT (7 Gy as a single dose) alone, or in combination with an αPD-L1 mAb. Median survival in the NT cohort was 15.5 days, which was not significantly improved by αPD-L1 mAb delivered 3qw as a monotherapy (median survival=18 days; FIGS. 6F and 6G). However, RT when delivered in combination with αPD-L1 mAb led to significantly improved survival when compared to either monotherapy alone, with >70% of mice undergoing complete therapeutic response.

SEQUENCE LISTING

SEQ ID NO: 1
EIVLTQSPGTLSLSPGERATLSCRASQRVSSSYLAWYQQKPGQAPRLLI
YDASSRATGIPDRFSGSGSGTDFTLTISRLEPEDFAVYYCQQYGSLPWT
FGQGTKVEIK
SEQ ID NO: 2
EVQLVESGGGLVQPGGSLRLSCAASGFTFSRYWMSWVRQAPGKGLEWVA
NIKQDGSEKYYVDSVKGRFTISRDNAKNSLYLOMNSLRAEDTAVYYCAR
EGGWFGELAFDYWGQGTLVTVSS
SEQ ID NO: 3-VH CDR1
GFTFSRYWMS
SEQ ID NO: 4-VH CDR2
NIKQDGSEKYYVDSVKG
SEQ ID NO: 5-VH CDR3
EGGWFGELAFDY
SEQ ID NO: 6-VL CDR1
RASQRVSSSYLA
SEQ ID NO: 7-VL CDR2
DASSRAT
SEQ ID NO: 8-VL CDR3
QQYGSLPWT

1: A method of extending progression-free survival (PFS) in a patient with unresectable non-small-cell lung cancer (NSCLC), the method comprising concurrently treating the patient with an anti-PD-L1 antibody and chemoradiation therapy.

2: The method of claim 1, wherein the anti-PD-L1 antibody is human.

3: The method of claim 1, wherein the chemoradiation therapy is platinum-based.

4: The method of claim 2, wherein the human anti-PD-L1 antibody comprises a light chain variable domain comprising the amino acid sequence of SEQ ID NO: 1 and a heavy chain variable domain comprising the amino acid sequence of SEQ ID NO: 2.

5: The method of claim 2, wherein the human anti-PD-L1 antibody comprises:
a VH CDR1 having the amino acid sequence of SEQ ID NO: 3; and
a VH CDR2 having the amino acid sequence of SEQ ID NO: 4; and
a VH CDR3 having the amino acid sequence of SEQ ID NO: 5; and
a VL CDR1 having the amino acid sequence of SEQ ID NO: 6; and
a VL CDR2 having the amino acid sequence of SEQ ID NO: 7; and
a VL CDR3 having the amino acid sequence of SEQ ID NO: 8.

6: The method of claim 2, wherein the human anti-PD-L1 antibody is durvalumab, avelumab, atezolizumab, or sugemalimab.

7: The method of claim 1, wherein treatment with the anti-PD-L1 antibody comprises administering 1500 mg of the anti-PD-L1 antibody to the patient intravenously every four weeks (Q4W).

8: The method of claim 1, wherein the unresectable NSCLC is stage III and/or is locally advanced.

9. (canceled)

10: The method of claim 1, wherein the anti-PD-L1 antibody is administered on the first day of chemoradiation therapy.

11: A method of increasing the overall response rate (ORR) in a patient with unresectable non-small-cell lung cancer (NSCLC), the method comprising concurrently treating the patient with an anti-PD-L1 antibody and chemoradiation therapy.

12: The method of claim 11, wherein the anti-PD-L1 antibody is human.

13: The method of claim 11, wherein the chemoradiation therapy is platinum-based.

14: The method of claim 12, wherein the human anti-PD-L1 antibody comprises a light chain variable domain comprising the amino acid sequence of SEQ ID NO: 1 and a heavy chain variable domain comprising the amino acid sequence of SEQ ID NO: 2.

15: The method of claim 12, wherein the human anti-PD-L1 antibody comprises:
a VH CDR1 having the amino acid sequence of SEQ ID NO: 3; and
a VH CDR2 having the amino acid sequence of SEQ ID NO: 4; and
a VH CDR3 having the amino acid sequence of SEQ ID NO: 5; and
a VL CDR1 having the amino acid sequence of SEQ ID NO: 6; and
a VL CDR2 having the amino acid sequence of SEQ ID NO: 7; and
a VL CDR3 having the amino acid sequence of SEQ ID NO: 8.

16: The method of claim 12, wherein the human anti-PD-L1 antibody is durvalumab, avelumab, atezolizumab, or sugemalimab.

17: The method of claim 11, wherein
treatment with the anti-PD-L1 antibody comprises administering 1500 mg of the anti-PD-L1 antibody to the patient intravenously every four weeks (Q4W).

18: The method of claim 11, wherein the unresectable NSCLC is stage III and/or locally advanced.

19. (canceled)

20: The method of claim 11, wherein the anti-PD-L1 antibody is administered on the first day of chemoradiation therapy.

21: A method of treating a patient with stage III unresectable non-small-cell lung cancer (NSCLC), the method comprising concurrently treating the patient with an anti-PD-L1 antibody and chemoradiation therapy.

22: The method of claim 21, wherein the anti-PD-L1 antibody is human.

23: The method of claim 21, wherein the chemoradiation therapy is platinum-based.

24: The method of claim 22, wherein the human anti-PD-L1 antibody comprises a light chain variable domain comprising the amino acid sequence of SEQ ID NO: 1 and a heavy chain variable domain comprising the amino acid sequence of SEQ ID NO: 2.

25: The method of claim 22, wherein the human anti-PD-L1 antibody comprises:
a VH CDR1 having the amino acid sequence of SEQ ID NO: 3; and
a VH CDR2 having the amino acid sequence of SEQ ID NO: 4; and
a VH CDR3 having the amino acid sequence of SEQ ID NO: 5; and
a VL CDR1 having the amino acid sequence of SEQ ID NO: 6; and
a VL CDR2 having the amino acid sequence of SEQ ID NO: 7; and
a VL CDR3 having the amino acid sequence of SEQ ID NO: 8.

26: The method of claim 22, wherein the anti-PD-L1 antibody is durvalumab, avelumab, atezolizumab, or sugemalimab.

27: The method of claim 21, wherein treatment with the anti-PD-L1 antibody comprises administering 1500 mg of the anti-PD-L1 antibody to the patient intravenously every four weeks (Q4W).

28: The method of claim 21, wherein the anti-PD-L1 antibody is administered on the first day of chemoradiation therapy.

29-43. (canceled)
